(19) United States
(12) Patent Application Publication
(10) Pub. No.: US 2007/0123857 A1
(43) Pub. Date: May 31, 2007

(54) Orthopaedic joint, device and associated method

(76) Inventors: Daren L. Deffenbaugh, Winona Lake, IN (US); Kyle D. Blatt, Pickerington, OH (US); Joseph G. Wyss, Fort Wayne, IN (US); Robert J. Schneider, Goshen, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/260,081

(22) Filed: Oct. 27, 2005

(51) Int. Cl. A61B 17/00

(52) U.S. Cl. 606/54

(57) ABSTRACT

An articulating joint for rigidly connecting a first object to a second object for use in orthopedics is provided. The joint includes a body and a first articulating member being selectively one of pivotably connected to and rigidly connected to the body. The first articulating member is connectable to the first object. The joint also includes a second articulating member being selectively one of pivotably connected to and rigidly connected to the body. The second articulating member is connectable to the second object. The first articulating member and second articulating member are adapted for simultaneous locking and unlocking to each other.

### CROSS-REFERENCE TO RELATED APPLICATIONS

Cross reference is made to the following applications: DEP5427 titled, “SUPPORT FOR LOCATING INSTRUUMENT GUIDES”, DEP5597USNP titled, “METHOD OF RESECTING BONE”, DEP5368USNP titled “TRAUMA JOINT, EXTERNAL FIXATOR AND ASSOCIATED METHOD” and DEP5558USNP titled “ORTHOPAEDIC INSTRUMENT JOINT, INSTRUMENT AND ASSOCIATED METHOD” filed concurrently herewith which are incorporated herein by reference.

### TECHNICAL FIELD OF THE INVENTION

The present invention relates generally to the field of orthopaedics, and more particularly, to a device for use in treating orthopaedic trauma.

### BACKGROUND OF THE INVENTION

The skeletal system includes many long bones that extend from the human torso. These long bones include the femur, fibula, tibia, humerus, radius and ulna. These long bones are particularly exposed to trauma from accidents, and as such often are fractured during such trauma and may be subject to complex devastating fractures.

Automobile accidents, for instance, are a common cause of trauma to long bones. In particular, the femur

Often the distal end or proximal portions of the long bone, for example, the femur and the tibia, are fractured into several components and must be realigned. Mechanical devices, commonly in the forms of pins, plates, screws, nails, wires and external devices are commonly used to attach fractured long bones. The pins, plates, wires, nails and screws are typically made of a durable material compatible to the human body, for example titanium, stainless steel or cobalt chromium.

Fractures of the long bone are typically secured into position by at least one of three possible techniques.

The first method is the use of intramedullary nails that are positioned in the intramedullary canal of those portions of the fractured bone.

The first method is the use of intramedullary nails that are positioned in the intramedullary canal of those portions of the fractured bone.

A second method of repairing fractured bones is the use of internal bone plates that are positioned under the soft tissue and on the exterior of the bone and bridges the fractured portion of the bone.

Another method of securing fractured bones in position is the use of external fixators. These external fixators have at least two general categories. In one category the fixator is generally linear with a first portion of the fixator to connect to a first fracture segment of the bone and a second fracture segment of the fixator to connect to the second fracture segment of the bone. A first series of bone screws or pins are first connected to the fixator and then into the first portion of the bone. Then a second series of screws or pins are connected to the fixator and then to the second fracture segment of the bone, thereby securing the first portion fracture segment of the bone to the second portion of the bone. These types of fixators use screws and pins that are connected to rigid internal frames and rely on the rigidity of the frame to assure that the fixation is secure. One such linear fixator is sold by DePuy Orthopaedics, Inc., Warsaw, Ind. and marketed as the DePuy ACE Align® Fixator.

Rigid pins are placed into the proximal portion of the fractured bone and pins are placed into the distal portion of the fractured bone. The linear fixator is then attached to the two sets of pins bridging the fracture site and holding the two bone segments in place.

A second method of external fixation is through the use of a ring type fixator that uses a series of spaced apart rings to secure the bone. For example, an upper ring and a lower ring are spaced apart by rods. A plurality of wires is placed through the long bone and is connected on each end of the long bone by the ring. The wires are then tensioned much as a spoke in a bicycle are tightened, thereby providing for a rigid structure to support the first fracture segment portion of the bone. Similarly, a plurality of wires are positioned through the second fracture segment of the bone and are secured to and tensioned by the lower ring to provide a rigid fixation of the second fracture segment of the bone bridging the fracture site.

Such external fixators that utilize the tension wire approach may also be used with rigid pins in combination with the wires.

When utilizing either pins or wires for the external fixator, it is desirable that the wires are as small in diameter as possible to minimize the damage to soft tissue and to the bone during the fixation process. Further, it is important that the wires and pins move through the body and particularly through the bone in a generally linear fashion, such that when tightened the wires do not cause undue stresses on the soft tissues, and, particularly, the bone. The wires and pins typically have a cutting edge on the leading portion of the pin or wire to assist in the movement of the pin or wire through the soft tissue and bone of the patient. The pins or wires are typically mounted to a power rotational tool that is utilized to drill the pin or wire through the body.

In the orthopedic reconstruction of a patient's bone and/or joint, particularly with respect to bone repair thereof, it is necessary to keep the repaired bone and/or joint in an immobilized and stable state during the healing process. This is accomplished by using a frame construct that typically includes many different fixation components. The various fixation components are utilized to build a fixation device for immobilizing the bone and/or joint. One such fixation component may be an immobilization platform or platform construct.

In the area of the foot and/or ankle, what is known as a foot frame is generally utilized. Current foot frames are typically of an open U-ring type. The open U-rings may comprise a single “horseshoe-shaped” frame or may include myriad pieces that must be assembled during and for use (known as a modular foot frame).

During the particular surgery, one or more wires, pins, or half pins as they are known in the art are implanted through particular bones of the bone/joint (e.g. the foot and/or ankle). These wires, olive wires, pins, or half pins (collectively, wires) are utilized to immobilize and/or apply compression to the particular and/or surrounding bones in order to create a proper healing environment. The wires themselves need to be externally fixed in order to create a desired compression result on the bone(s) and/or joint(s). This is currently accomplished by tying the wires to wire/rod nuts on the various components of the open U-ring foot frame. These systems, however, suffer problems with respect to being able to achieve the desired compression results, e.g. the ability to adequately externally fix the wires and provide controlled compression. This can lead to instability problems. Moreover, it is difficult to achieve accurate in-plane compression with current fixation devices.

In order to resolve these problems, the prior art bends the transverse wires from the ankle/foot, then tensions the bent wires to achieve compression. This is known as walking the wires. The bent and tensioned wires are then attached to the open U-frame. Tensioning bent wires, however, does not provide a controlled or measurable amount of compression on the desired area of the ankle/foot.

With respect to orthopaedic surgery and particularly with respect to the foot and/or ankle, the surgical area (ankle/foot area) is exposed. It is, thus, necessary in some respects to protect the particular area (ankle/foot). Prior art fixation devices utilize an additional ring positioned inferior to the foot frame to protect the bottom of the foot. This technique is time consuming and costly.

Osteoarthritis and rheumatoid arthritis are common afflictions of the joints of the human body. The ankle is one of the many joints, which may be susceptible to osteoarthritis. Arthrodesis has been an accepted treatment for painful osteoarthritis and rheumatoid arthritis of the ankle and the subtalor joints for many years. In the most common of arthrodesis, the talus, tibia and calcaneus are fused together. Such a procedure is commonly known as an ankle fusion. Another less common treatment for arthritis of the ankle is total ankle arthroplasty. Total ankle arthroplasty can be described in greater detail in U.S. Pat. No. 5,326,365 to Alvine, hereby incorporated by reference in its entirety.

A portion of the ankle fusion procedure is to resect the distal tibia and the proximal talus. The resected surface of the distal tibia and the proximal talus are then fused together. The tibia and talus may be fused together using any of a combination of bone plates, bone screws, and intramedullary nails. To perform the tibia and talus resections, the ankle joint is distracted approximately one centimeter. While in this distracted condition, the tibia and talus are resected. The joint is then relaxed and then the tibia and talus are fused.

The resection of the tibia and talus are typically performed utilizing a saw blade that is held in the surgeon's hand and the resection is performed free hand. The free-hand resection of the tibia and talus has several problems. One problem with the current free-hand method of resection is the danger of over resection of the joint surfaces. If too great a resection is performed, the ankle joint height is compromised. The patient then may have a resected leg length that is unacceptably shorter than the unfused leg length. Another problem with the present free-hand method of resecting the tibia and talus is that fore and hind foot alignment may be inaccurate. Alignment is very important because a fused ankle has only a limited degree of flexion. Excessive dorsal flexion or plantar flexion may cause gait problems or patient pain.

In utilizing external fixators, the position of the pins, which engage the bones, is often critical. Thus, in prior art, fixation devices have included a locking mechanism to provide for an articulating adjustment between a first portion and a second portion of the fixator device. To provide for sufficient adjustment of the different portions of the fixator device a plurality of, for example two or more, separate locking mechanisms are utilized to provide for the amount of adjustment required to provide for the proper positioning of the pins related to an external fixator.

For example, some external fixators are particularly troublesome to properly adjust the position of the first set of fixator pins with respect to the second set of fixator pins. One such application is related to external fixators for foot positioners. For proper foot positioning for an external fixator, the posterior and anterior positioning, inversion and eversion, as well as dorsal and planar flexion must be properly positioned for proper bone resection for ankle fusion or for total ankle arthroplasty. The proper positioning of the feet with respect to the tibia can be quite troublesome. In fact, many adjustments may be necessary for the various locking mechanisms to establish the proper positioning of the bones in the feet.

Attempts have been made in the prior art to provide for the adjustment of the various positions of an external fixator. Such distraction advice includes a series of locked ball joints to provide for the motion. Such distractors require three, four or more adjustments to lock the ball joints.

U.S. Pat. No. 6,036,691 provides two separate cam locks for two separate ball joints. The distal member of the foot positioner allows dorsal and planar flexion but does not provide for inversion and eversion.

Another prior art patent, U.S. Pat. No. 6,461358 B1 also attempts to provide for positioning of the foot.

Distractors may also be included in an external fixator. Such distractors are often used for external fixators for preparation of ankle fusion or total ankle arthroplasty. Resection cuts are performed with the use of the distractor device to provide for proper leg length.

### SUMMARY OF THE INVENTION

The present invention serves as an external fixator for use in the distraction of the ankle. The present invention may also be used as an external fixator for use in distractions of other portions of the skeleton. The present invention may also serve as a portion of an external fixator for use with cutting blocks to provide for resection cuts of bone for use, for example, in preparing bones for orthopedic implants.

The proximal member of the external fixator consists of a body that is attached to the tibia with pins and contains a distraction device that translates the medial and distal members. One embodiment includes a proximal member attached to a medial member with a lockable ball joint. The distal member attaches to the opposed end of the medial member with a lockable ball joint.

The present invention may include a locking cam mechanism that locks the proximal and distal member of the ball joint simultaneously. To provide for radiological measurements, the distal member may be composed of a radiolucent material that is fixed to the ankle with pins. The distal member allows for inversion and eversion of the anklebone segments along with dorsal and planar flexion.

The locking fixation device of the present invention provides for a single locking mechanism for two distinct ball joints. The mechanism allows the surgeon to lock the distraction device with a single mechanism instead of locking with several different modifications. The use of a single mechanism saves the surgeon time by reducing the number of adjustments required to properly position, for example, the foot. The distal member of the foot positioner allows the position of the pins or wires to be fully customized for the proper orientation of the foot. The fixation device includes posterior and anterior movement of the wire clamps along with dorsal and planar reflection. The distal member also allows rotation of the wire clamp assembly inversely and eversely. This allows the wires and foot to be adjusted any way the surgeon wants to position it.

The foot positioner of the present invention includes a locking articulation member, which locks two spherical members simultaneously. The locking articulation joint includes a cam that translates two bearings in opposite direction to lock two separate spherical members. The actuator or cam rotates in a slit or channel that allows both spherical members to be locked with an equal amount of force. The actuator allows translation of the bearing with a line-to-line action that prevents binding of the bearings while locking.

The cam lock feature of the present invention provides enhanced performance and reduced time required to lock the external fixator. The distal portions of the foot positioner are composed of carbon fiber bars and nylon wire clamps. The carbon fiber bars and nylon wire clamps are available as part of the TempFix® External Fixator product line available from DePuy Orthopaedics, Inc. The carbon fiber bars are connected to aluminum rotating clamps that are tightened using bolts. The bolts can be loosened to allow rotation of the wire clamp assemblies in any orientation required by the surgeon. The wire clamps can be moved along the carbon fiber bar to be customized for every individual angle.

The articulating joint for use in the external fixating device of the present invention includes a body. The body includes a central cavity and opposed caps. As an actuator rotates in a slitted hole formed in the body, the actuator translates two pistons. The actuator is rotated until the pistons push into the articulating members. The articulating members are locked when they press against the caps. This occurs on both sides of the actuating joints simultaneously.

The actuator includes a shaft that is fitted loosely in a slit. The slit in the body allows the actuator to rotate and lock with an equal amount of force. The purpose of the slit is to allow for any differences in tolerances so that both articulating members lock simultaneously every time. The actuator includes a cam, which cooperates with a piston. The cam includes a rounded cut out that follows an elliptical path. The cam is designed to fit with the spherical radius of the piston. The cam and spherical radius on the piston allows the piston to be translated without binding.

The distal portion of the foot positioner allows several different adjustments to allow for any type of orientation of the foot. It incorporates the same ideas as that of the TempFix® External Fixation Platform available from DePuy Orthopaedics, Inc. The wire clamps of the foot positioner can be moved along the vertical carbon fiber bar allowing proper placement of the pins in the ankle. The bar clamp may be tightened by a bolt to allow inversion and eversion by allowing the bar to rotate about the bar clamp. A bar end clamp that mates with a bar clamp is connected by the use of a bolt. Loosening the bolt allows dorsal and planar flexion of the ankle. All of these adjustments allow positioning of the pins and orientation of the foot in any possible position.

According to one embodiment of the present invention, there is provided a device for use in an external fixator for use in trauma surgery for rigidly connecting a first object to a second object. The device includes a body and a first articulating member for connecting the device to the first object. The first articulating member is lockable and un-lockable to the body to selectively provide articulation with and rigid connection to the body. The device further includes a second articulating member for connecting the device to the second object. The second articulating member is lockable and un-lockable to the body to selectively provide articulation with and rigid connection to the body. The body, the first articulating member and the second articulating member are adapted for simultaneous locking and unlocking to each other.

According to another embodiment of the present invention there is provided a device for use in an instrument for use in preparing bone for receiving an orthopaedic implant for use in orthopaedic surgery for rigidly connecting a first object to a second object. The device includes a body and a first articulating member for connecting the device to the first object. The first articulating member is lockable and un-lockable to the body to selectively provide articulation with and rigid connection to the body. The device further includes a second articulating member for connecting the device to the second object. The second articulating member is lockable and un-lockable to the body to selectively provide articulation with and rigid connection to the body. The body, the first articulating member and the second articulating member are adapted for simultaneous locking and unlocking to each other.

According to another embodiment of the present invention there is provided an articulating joint for rigidly connecting a first object to a second object for use in orthopedics. The joint includes a body and a first articulating member being one of pivotably and rigidly connected to the body. The first articulating member is connected to the first object. The joint also includes a second articulating member being selectively one of pivotably connected to and rigidly connected to the body. The second articulating member is connectable to the second object. The first and second articulating members are adapted for simultaneous locking and unlocking to each other.

According to another embodiment of the present invention there is provided an external fixator for use in trauma surgery for rigidly connecting a first portion of bone to a second portion of bone. The fixator includes a body and a first articulating member for connecting the device to the first object. The first articulating member is lockable and un-lockable to the body to selectively provide articulation with and rigid connection to the body. The device further includes a second articulating member for connecting the device to the second object. The second articulating member is lockable and un-lockable to the body to selectively provide articulation with and rigid connection to the body. The body, the first articulating second and the second articulating member are adapted for simultaneous locking and unlocking to each other.

According to another embodiment of the present invention there is a method for performing orthopaedic surgery. The method includes the steps of providing a cutting block for attachment to a bone. The cutting block includes an articulating joint for rigidly connecting the cutting block to the bone. The joint includes a body and a first articulating member that is selectively pivotably connected to or rigidly connected to the body. The first articulating member is connectable to the first object. The joint also includes a second articulating member that is selectively pivotably connected to or rigidly connected to the body. The second articulating member is connectable to the second object, the first and second articulating member being adapted for simultaneous locking and unlocking to each other. The method also includes the steps of securing the cutting block to the bone, unlocking the articulating joint, and aligning the cutting block to provide an accurate cutting of the bone. The method further includes the steps of locking the articulation joint and cutting the bone.

According to another embodiment of the present invention there is provided a method for rigidly securing a first portion of bone to a second portion of bone during trauma surgery. The method includes the steps of providing an external fixator for attachment to the first portion of bone and to the second portion of bone. The external fixator includes an articulating joint for rigidly connecting the external fixator to the first portion of bone and to the second portion of bone. The joint includes a body, a first articulating member that is selectively either pivotably connected to or rigidly connected to the body. The first articulating member is connectable to the first object. The joint also includes a second articulating member that is selectively either pivotably connected to or rigidly connected to the body. The second articulating member is connectable to the second object. The first and second articulating member are adapted for simultaneous locking and unlocking to each other. The method also includes the steps of securing the external fixator to the first portion of bone and unlocking the articulating joint. The method further includes the step of aligning the first portion of bone and the second portion of bone to provide proper orthopaedic alignment. The method also includes the steps of securing the external fixator to the second portion of bone and locking the articulation joint.

According to yet another embodiment of the present invention there is provided a method for rigidly securing a first portion of bone to a second portion of bone during orthopaedic surgery. The method includes the step of providing a device for attachment to a bone. The device includes an articulating joint for rigidly connecting the device to the first portion of bone and to the second portion of bone. The articulating joint includes a body and a first articulating member. The first articulating member is selectively one of pivotably connected to and rigidly connected to the body. The first articulating member is connectable to the first object. The articulating joint also includes a second articulating member. The second articulating member is selectively pivotably connected to or rigidly connected to the body. The second articulating member is connectable to the second object. The first articulating member and second articulating member are adapted for simultaneous locking and unlocking to each other. The method also includes the steps of securing the device to the first portion of bone, unlocking the articulating joint, aligning the first portion of bone and the second portion of bone to provide proper orthopaedic alignment, securing the device to the second portion of bone, and locking the articulation joint.

According to yet another embodiment of the present invention there is provided a device for securing a first bone portion to a second portion. The device includes a first object for securement to the first bone portion and a second object for securement to the second bone portion. The device further includes an articulating joint for rigidly connecting the first object to the second object. The articulating joint has a first articulating member that is selectively pivotably connected to or rigidly connected to the body. The first articulating member is connectable to the first object. The articulating joint also has a second articulating member that is selectively pivotably connected to or rigidly connected to the body. The second articulating member is connectable to the second object. The first articulating member and second articulating member are adapted for simultaneous locking and unlocking to each other.

The technical advantages of the present invention include the ability to permit two ball joints to be locked simultaneously. For example, according to one aspect of the present invention an articulating joint for rigidly connecting a first object to a second object for use in orthopedics is provided. The joint includes a body and a first and second articulating member. The first articulating member is selectively pivotably connected or rigidly connected to the body. The first articulating member is connectable to the first object. The second articulating member is selectively pivotably connected or rigidly connected to the body. The second articulating member is connectable to the second object. The first articulating member and the second articulating member are adapted for simultaneous locking and unlocking to each other. Thus, the present invention provides for two ball joints to be locked simultaneously.

The technical advantages of the present invention further include the ability to save the surgeon time in utilizing external fixators. For example, according to another aspect of the present invention, a device for use in an external fixator for use in trauma surgery for connecting a first object to a second object is provided. The device includes a body, as well as first and second articulating members. The first articulating member connects the device to the second object and is lockable and un-lockable to the body to selectively provide articulation with and rigid connection to the body. The second articulating member is used to connect the device to the second object. The second articulating member is lockable and un-lockable to the body to selectively provide articulation with and rigid connection to the body. The body and the first and second articulating members are adapted for simultaneously locking and unlocking to each other. Thus, the present invention provides for the saving of surgeons' time by permitting two ball joints to be simultaneously locked.

The technical advantages of the present invention further include the ability to reduce the number of adjustments required. For example, according to yet another aspect of the present invention, a device for use in an instrument for preparing bone is provided. The device includes a body and first and second articulating members. The first articulating member is used to connect the device to the first object. The first articulating member is lockable and un-lockable to selectively provide articulation with the body. The second articulating member is used to connect the device to the second object. The second articulating member is lockable and un-lockable to the body to provide articulation with and rigid connection to the body. The body, the first articulating member and the second articulating member are adapted for simultaneously locking and unlocking to each other. Thus, the present invention provides for a reduction in the number of adjustments required in that the two separate joints can be simultaneously locked and thereby the surgeon can place the bone pins in the proper position by having more ability to properly orient the external fixator holding the pins.

The technical advantages of the present invention also include the ability to provide another form of rotation with the foot positioner. For example, according to yet another aspect of the present invention, a device for use as an external fixator for use in trauma surgery for connecting the tibia to the bones of the foot include a body, and first and second articulating members. The first articulating member is used to connect the device to the first object. The first articulating member is lockable and un-lockable to the body to provide articulation with and rigid connection to the body. The second articulating member is used to connect the device to the second object and is lockable and un-lockable to the body to selectively provide articulation with and rigid connection to the body. The body and the first and second articulating members are adapted for simultaneously locking and unlocking to each other. Thus, the present invention provides another form of rotation with a foot positioner by permitting the surgeon to orient the foot in any direction by utilizing the two ball joints and then locking them together simultaneously.

The technical advantages of the present invention further include the ability to permit inversion and eversion rotation simultaneously with the ability to permit the positioning of the dorsal and plantar flexion. For example, according to yet another aspect of the present invention, a device for use in an external fixator for use in trauma surgery for performing an ankle fusion or ankle arthroplasty is provided. The device includes a body as well as first and second articulating members. The articulating members are selectively positioned in a locked and unlocked position with the first and second articulating members being adapted for simultaneously locking and unlocking to each other. The surgeon may adjust the foot positioner in any orientation including inversion and eversion, as well as dorsal and planar flexion easily and then lock the foot positioner in that position by utilizing the locking device with the two articulating members.

Other technical advantages of the present invention will be readily apparent to one skilled in the art from the following figures, descriptions and claims.

### BRIEF DESCRIPTION OF THE DRAWINGS

FIG. 1 is a perspective view of an articulating joint for rigidly connecting a first object to a second object for use in orthopaedics in accordance to an embodiment of the present invention;

FIG. 2 is a plan view, partially in cross section, of the articulating joint of FIG. 1;

FIG. 2A is a partial plan view, partially in cross section, of the actuator of the articulating joint of FIG. 1;

FIG. 2B is a partial plan view, partially in cross section, of the actuator of the articulating joint of FIG. 1;

FIG. 2C is a partial plan view of another embodiment of the present invention in the form of an actuator with a circular opening;

FIG. 3 is a partial cross sectional view of the articulating joint of FIG. 1;

FIG. 3A is a partial plan view, partially in cross-section of another embodiment of the present invention in the form of an actuator with a wedge shape;

FIG. 4 is a partial plan view, partially in cross section, of the actuator of another embodiment of the articulating joint the present invention showing a body with a circular body transverse opening;

FIG. 5 is a partial plan view, partially in cross section, of the actuator of another embodiment of the articulating joint the present invention showing an actuator with a wedge;

FIG. 6 is a plan view of a fixator incorporating an articulating joint for rigidly connecting a first object to a second object for use in orthopedics in accordance with yet another embodiment of the present invention;

FIG. 7 is a plan view of the articulating joint of the fixator of FIG. 6;

FIG. 8 is a cross-sectional of FIG. 7 along the line 8-8 in the direction of the arrows;

FIG. 9 is a plan view of the body of the articulating joint of FIG. 7;

FIG. 10 is a top view of the body of FIG. 8 of the articulating joint of FIG. 7;

FIG. 11 is a plan view of the articulating member of the articulating joint of FIG. 7;

FIG. 12 is a cross-sectional view of FIG. 11 along the line 12-12 in the direction of the arrows;

FIG. 13 is a plan view of the piston of the articulating joint of FIG. 7;

FIG. 14 is a cross-sectional view of FIG. 13 along the line 14-14 in the direction of the arrows;

FIG. 15 is a bottom view of the piston of FIG. 12 of the articulating joint of FIG. 7;

FIG. 16 is a plan view of the cam of the articulating joint of FIG. 7;

FIG. 17 is a cross-sectional view of FIG. 16 along the line 17-17 in the direction of the arrows;

FIG. 18 is a side view of the cam of FIG. 16 of the articulating joint of FIG. 7;

FIG. 19 is a plan view of an external fixator for use in ankle fusion shown with an ankle fusion cutting guide in position on the body of a patient in accordance to yet another embodiment of the present invention;

FIG. 20 is a partial plan view of the external fixator of FIG. 19 showing the portion of the fixator around the foot in greater detail;

FIG. 21 is a partial plan view of an external fixator of another embodiment of the device of the present invention having a U-shaped bar portion surrounding the foot;

FIG. 22 is a partial perspective view of the external fixator of FIG. 19 showing the portion of the fixator around the actuating joint and the distractor in greater detail;

FIG. 23 is a plan view of the distractor pin of the distractor of the external fixator of FIG. 22;

FIG. 23A is a cross-sectional view of FIG. 23 along the line 23A-23A in the direction of the arrows;

FIG. 24 is a plan view of the distractor screw of the distractor of the external fixator of FIG. 22

FIG. 25 is an enlarged partial perspective view of the external fixator of FIG. 22;

FIG. 26 is another further enlarged partial perspective view of the external fixator of FIG. 22;

FIG. 27 is a plan view of the connector of the external fixator of FIG. 22;

FIG. 28 is an end view of the connector of the external fixator of FIG. 22;

FIG. 29 is a plan view of the bar of the external fixator of FIG. 22;

FIG. 30 is a partial plan view of an external fixator of another embodiment of the device of the present invention having a bar with pins protruding transversely through the bar;

FIG. 31 is a plan view of the bar/periphery half of the bar clamp of the external fixator of FIG. 22;

FIG. 32 is a side view of the bar/periphery half of FIG. 31;

FIG. 33 is an end view of the bar/periphery half of FIG. 31;

FIG. 34 is a plan view of the bar end half of the bar clamp of the external fixator of FIG. 22;

FIG. 35 is a side view of the bar end half of FIG. 34;

FIG. 36 is a plan view of the bar clamp half of the bar-pin clamp of the external fixator of FIG. 22;

FIG. 37 is a side view of the bar clamp half of FIG. 36;

FIG. 38 is a perspective view of the pin clamp half of the bar-pin clamp of the external fixator of FIG. 22;

FIG. 39 is a perspective view of a spacer for use with the pin clamp half of the bar-pin clamp of the external fixator of FIG. 22;

FIG. 40 is a plan view of a fused ankle that may be prepared with the external fixator of FIG. 19;

FIG. 41 is a plan view of an external fixator for use in ankle arthroplasty shown with an ankle arthroplasty cutting guide in position on the body of a patient in accordance to yet another embodiment of the present invention;

FIG. 42 is a plan view of an ankle implant for use in an ankle that may be prepared with the external fixator of FIG. 41;

FIG. 43 is a plan view of an external fixator for use around the knee of a patient in accordance to yet another embodiment of the present invention;

FIG. 44 is a plan view of an external fixator for use around the elbow of a patient in accordance to yet another embodiment of the present invention;

FIG. 45 is a plan view of an external fixator for use in knee arthroplasty shown with a tibial knee arthroplasty cutting guide in position on the body of a patient in accordance to yet another embodiment of the present invention;

FIG. 46 is a plan view of an external fixator for use in knee arthroplasty shown with a femoral knee arthroplasty cutting guide in position on the body of a patient in accordance to yet another embodiment of the present invention;

FIG. 47 is a plan view of an external fixator for use in knee arthroplasty shown with a femoral hip arthroplasty cutting guide in position on the body of a patient in accordance to yet another embodiment of the present invention;

FIG. 48 is a perspective view of an articulating joint for rigidly connecting a first object to a second object for use in orthopaedics in accordance to yet another embodiment of the present invention having a telescoping two piece body;

FIG. 49 is a plan view, partially in cross section, of the articulating joint of FIG. 48, with the joint in the locked position;

FIG. 50 is a plan view, partially in cross section, of the articulating joint of FIG. 48, with the joint in the unlocked position;

FIG. 51 is a partial plan view, partially in cross section, of the articulating joint of FIG. 48 showing the articulating member and the body in greater detail;

FIG. 52 is a plan view, partially in cross section, of an articulating joint for rigidly connecting a first object to a second object for use in orthopaedics in accordance with a further embodiment of the present invention having a ratchet mechanism;

FIG. 52A is a cross-sectional view of FIG. 52 along the line 52A-52A in the direction of the arrows;

FIG. 52B is a cross-sectional view of FIG. 52 along the line 52B-52B in the direction of the arrows;

FIG. 53 is a plan view, partially in cross section, of an articulating joint for rigidly connecting a first object to a second object for use in orthopedics in accordance with a further embodiment of the present invention having cylindrical articulating portions of the articulating members;

FIG. 54 is a top view, partially in cross-section, of the joint of FIG. 53;

FIG. 55 is a partial top view, partially in cross-section, of the joint of FIG. 53;

FIG. 56 is a flow chart for a method of performing trauma surgery in accordance to yet another embodiment of the present invention; and

FIG. 57 is a flow chart for a method for performing orthopaedic surgery in accordance to another embodiment of the present invention.

Corresponding reference characters indicate corresponding parts throughout the several views. Like reference characters tend to indicate like parts throughout the several views.

### DETAILED DESCRIPTION OF THE INVENTION

Embodiments of the present invention and the advantages thereof are best understood by referring to the following descriptions and drawings, wherein like numerals are used for like and corresponding parts of the drawings.

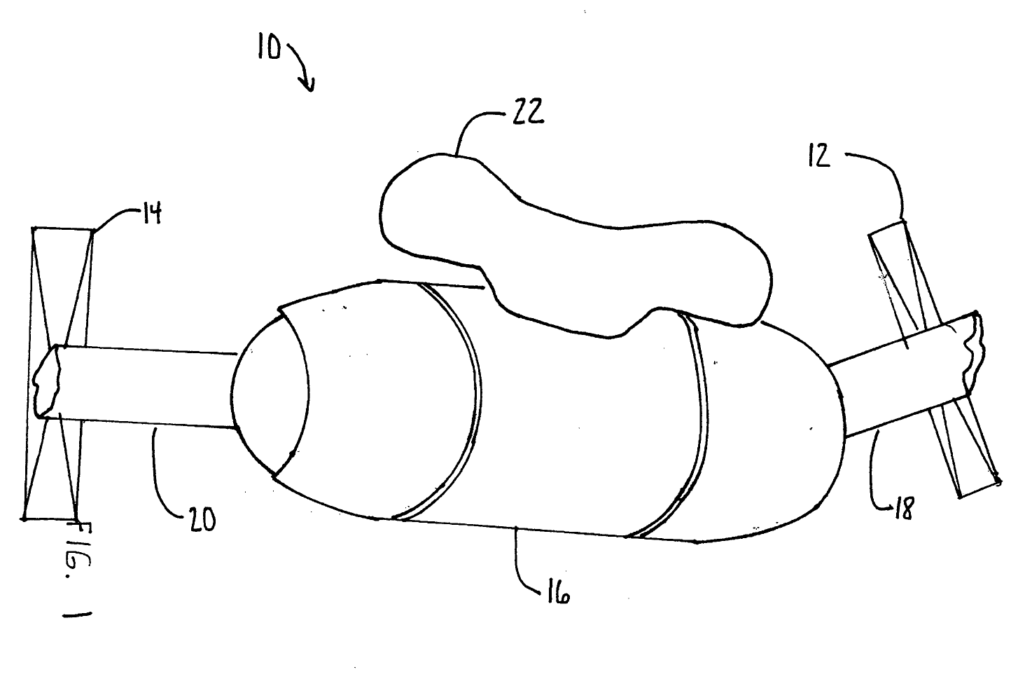

According to the present invention and referring now to FIG. 1 an articulating joint 10 is shown for rigidly connecting a first object 12 to a second object 14 for use in orthopedics. The articulating joint 10 includes a body 16 as well as a first articulating member 18. The first articulating member 18 is selectively either pivotably connected to or rigidly connected to the body 16. The first articulating member 18 is connectable to the first object 12.

The articulating joint 10 further includes a second articulating member 20. The second articulating member 20 is selectively either pivotably connected to or rigidly connected to the body 16. The second articulating member 20 is connectable to the second object 14. The first articulating member 18 and the second articulating member 20 are adapted for simultaneously locking and unlocking to each other.

The articulating joint 10 as shown in FIG. 1 may further include an actuator 22. The actuator is operably connected to the first articulating member 18 and to the second articulating member 20. The actuator 22 simultaneously locks and unlocks the first articulating member 18 and the second articulating member 20.

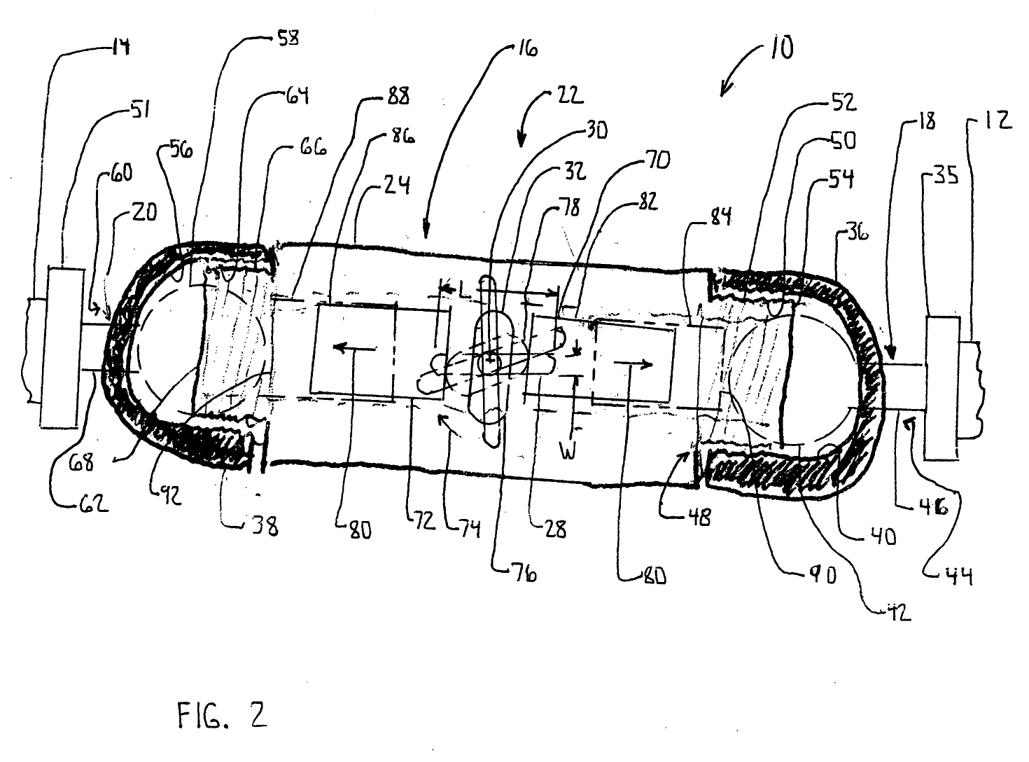

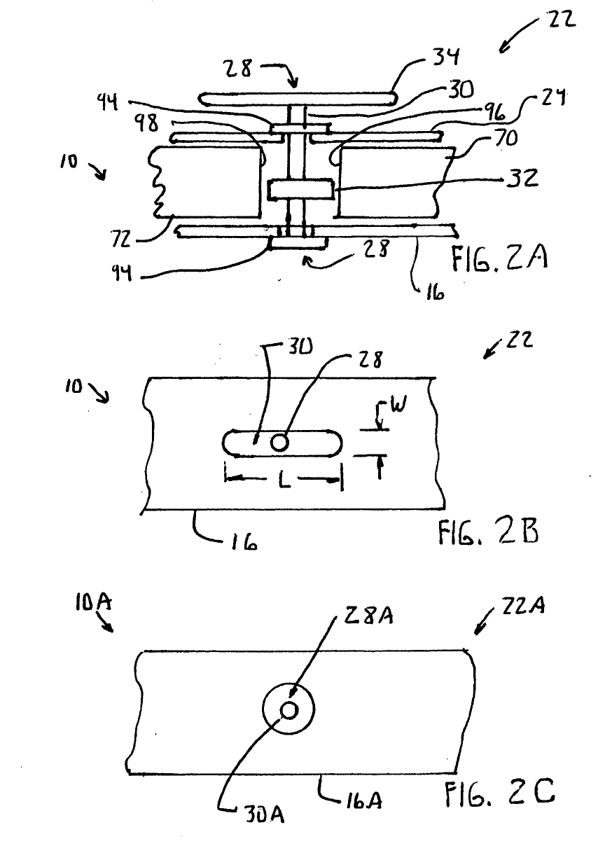

Referring now to FIG. 2, the articulating joint 10 is shown in greater detail. The articulating joint 10 includes the body 16. The body 16 may have any suitable shape capable of supporting the actuator 22 and for assisting to transfer motion from the actuator 22 to the first articulating member 18 as well as to the second articulating member 20.

For example and as shown in FIG. 2, the body 16 may be in the form of a generally tubular cylindrical member. The body 16 may include a generally cylindrical outer periphery 24 and a generally cylindrical bore 26. Bore 26 may as shown in FIG. 2 be concentric with outer periphery 24.

As shown in FIG. 2, the actuator 22 is mounted to the body 16. For example and as shown in FIG. 2, the body 16 may include a body transverse opening 28 through which shaft 30 of the actuator 22 is rotatably fitted. Preferably, to accommodate component tolerances and the resultant tolerance stack of the components of the articulating joint 10, the body transverse opening 28 may be sized to provide additional clearance between the body transverse opening 28 and the shaft 30. The clearance accommodates the tolerances so that the shaft 30 is not limited in its motion axially by the body 16.

For example and as shown in FIG. 2, the body transverse opening 28 may be oval. For example and as shown in FIG. 2, the body transverse opening 28 may be defined by an opening length L which is substantially greater than the opening width W. The opening length L is made sufficiently larger than diameter D of the shaft 30 such that the shaft 30 does not impinge upon the body 16.

The actuator 22 includes a feature 32 in the form of, for example, a cam for transferring force from the shaft 30 to the articulating members 18 and 20. The actuator 22 may, as shown in FIG. 1 further include a handle 34 for locking and releasing the articulating joint 10.

It should be appreciated that the body 16 may fully restrain the articulating members 18 and 20. It should also be appreciated that the joint 10 may, for simplicity, include restraining features in addition to the body 16. For example, the articulating joint 10 may further include a first cap 36 and an opposed second cap 38. Caps 36 and 38 may have any suitable shape capable of containing the articulating members 18 and 20 within the joint 10. For example and as shown in FIG. 2, the first cap 36 includes a concave inner-periphery 40 for cooperation with spherically shaped portion 42 of the first articulating member 18. The first cap 36 further defines a first cap member opening 44 for permitting stem portion 46 of the first articulating member 18 to pass therethrough.

The first cap 36 further defines the first cap body opening 48 for receiving the body 16. The first cap 36 may be secured to the body 16 in any suitable way, for example, by a series of pins, a groove and lip, or, as shown in FIG. 2, by internal threads 50 formed on the first cap 36 adjacent the first cap body opening 48. The internal threads 50 of the first cap 36 matingly engage external threads 52 formed on first hub 54 of the body 16.

The second cap 38 is similar to the first cap 36 and includes a concave inner-periphery 56 for cooperation with spherically shaped portion 58 of the second articulating member 20. The second cap 38 includes a second cap member opening 60 for passage of the stem portion 62 of the second articulating member 20. The inner periphery 56 of the second cap 38 includes internal threads 64, which mate with external threads 66 formed on hub 68 of the body 16.

The articulating joint 10 of the present invention may include a number of design alternatives to transfer the motion from the actuator 22 to the articulating members 18 and 20. For example, the articulating joint 10 may include a first piston 70 for positioning within the bore 26 of the body 16 as well as a second piston 72 likewise positioned in the bore 26 of the body 16. The pistons 70 and 72 serve to transfer motion from the cam 32 to the articulating members 18 and 20 respectively. For example and as shown in FIG. 2, as the cam 32 is rotated in the direction of arrow 74, the cam 32 moves from first position 76 to second position 78 as shown in phantom.

As the cam 32 moves from first position 76 to second position 78, the cam serves to advance the first piston 70 and second piston 72 in opposed directions in the directions of arrows 80. In particular, the first piston 70 advances from first position 82 (as shown in solid) to second position 84 (as shown in phantom). Similarly the second piston 72 advances from first position 86 (as shown in solid) to second position 88 (as shown in phantom).

When the first piston 70 and the second piston 72 are in their second positions, the outer face 90 of the first piston 70 becomes in locking engagement with the spherically shaped portion 42 of the first articulating member 18 locking it into position. Similarly and simultaneously, the second piston 72, when in its second position 88, provides for outer face 92 of the second piston 72 to be in locked engagement with spherically shaped portion 58 of the second articulating member 20. Thereby, the second piston 72 locks the second articulating member 20. Thus, the articulating joint 10 of the present invention provides for simultaneous locking of the first articulating member 18 and the second articulating member 20 with respect to the body 16 by the actuation of the actuator 22.

The outer face 90 of the first piston 70 and the outer face 92 of the second piston 72 may have, for example, concave surfaces to mate with the spherically shaped portions 42 and 58 of the first articulating member 18 and second articulating member 20, respectively. The concave surfaces provide for increased contact and superior locking of the articulating members 18 and 20.

Referring now to FIGS. 2A and 2B, the actuator 22 is shown in greater detail. As shown in FIG. 2A, the actuator 22 includes the shaft 30, which is slidably positioned in body transverse opening 28, which is positioned normal to outer periphery 24 of the body 16. The shaft 30 may be secured to the body 16 by a pair of fasteners 94 secured to the outer periphery 24 of the body 16, as well as to the shaft 30. Inner faces 96 and 98 of the first piston 70 and second piston 72, respectively, may as shown in FIG. 2A be convex to provide for a smooth movement of the cam 32 with respect to the pistons 70 and 72.

Referring now to FIG. 2B, the body transverse opening 28 may be defined by a width W closely conforming to the shaft 30 as well as a length L which is substantially larger than the width W and provides for variations in the accuracy of the components and their resulting tolerance stack so that the first piston 70 and the second piston 72 engage simultaneously with the first articulating member 18 and the second articulating member 20, respectively.

Referring now to FIG. 2C, yet another embodiment of the present invention is shown as articulating joint 10C. The articulating joint 10C is similar to the articulating joint 10 of FIG. 2 except that the articulating joint 10C includes an opening 28A in the body 16A of the joint 10A that is circular rather than elongate or oval. The opening 28A in the body 16A as shown in FIG. 2C is substantially larger than the shaft 30A such that the variations in tolerance may still permit the articulating members to simultaneously lock.

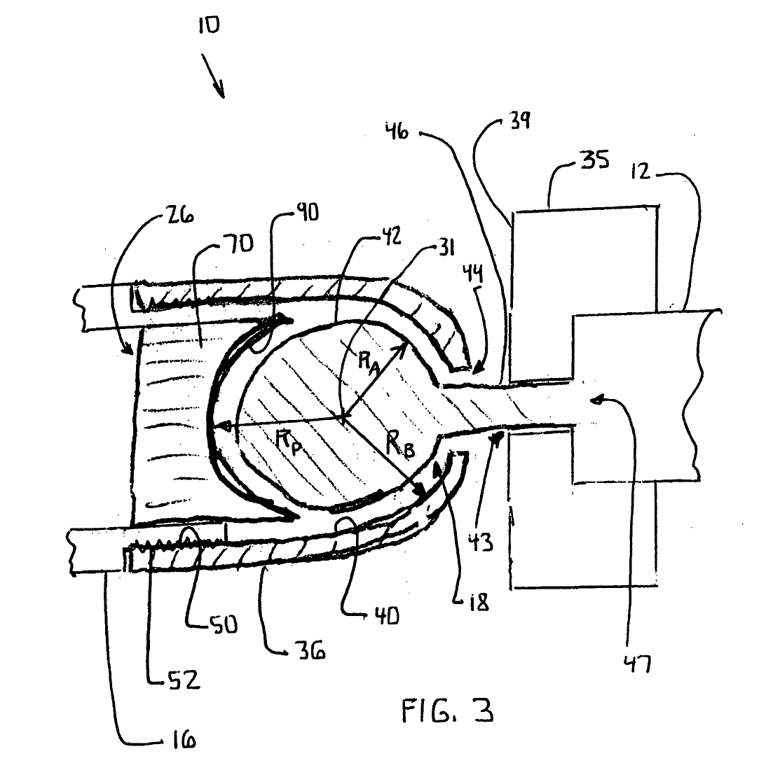

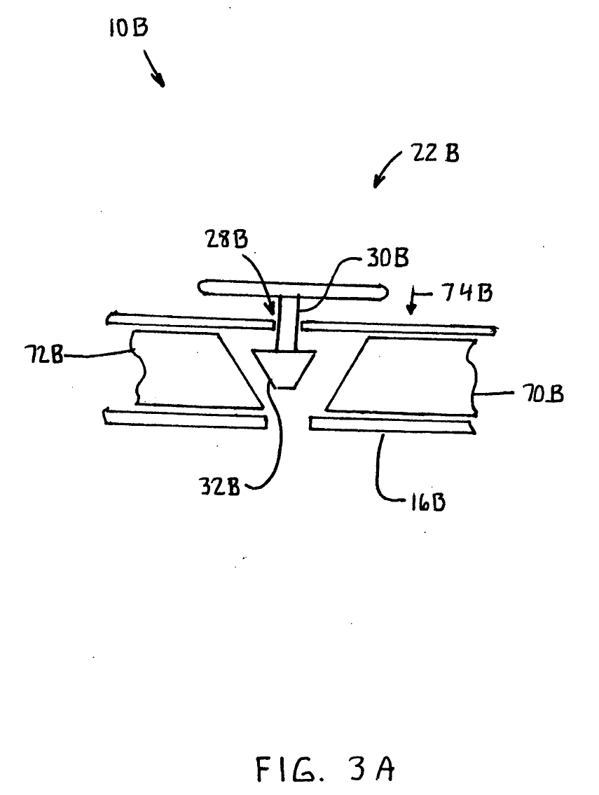

Referring now to FIG. 3, the first end of the articulating joint 10 is shown in greater detail. As shown in FIG. 3, the first cap 36 extends from body 16 and is threadably connected to the body 16 by internal threads 50 formed in the first cap 36, which mate with external threads 52 formed on the body 16. First piston 70 is slidably fitted in bore 26 of the body 16 and engages the spherically shaped portion 42 of the first articulating member 18.

Preferably and as shown in FIG. 3, outer face 90 of piston 70 has a concave shape to mate with the spherically shaped portion 42 of the first articulating member 18. The spherically shaped portion 42 of the first articulating member 18 also matingly fits with inner-periphery 40 of the first cap 36. The inner-periphery 40 of the first cap 36 may, as is shown in FIG. 3, have a concave shape, for example, a spherical shape. The first articulating member 18 also includes stem portion 46 that extends from the spherically shaped portion 42 and passes through first cap opening 44 formed in the first cap 36.

For example, and as shown in FIG. 3, the spherically shaped portion 42 of the first articulating member 18 is defined by a radius RA extending from origin 31. Similarly, the outer face 90 of piston 70 is defined by radius RP extending from the origin 31. Similarly, the inner-periphery 40 of the cap 36 is defined by radius RB extending from origin 31. As shown in FIG. 3, the radius RA of the spherically shaped portion 42 of the first articulating portion 18 is slightly smaller than the radii RP and RB of the piston 70 and cap 36 respectively.

According to the present invention the articulating joint 10 is connectable to the first object 12 as well as the second object 14 (see FIG. 2). The objects 12 and 14 may be connected to the articulating joint in any suitable manner. For example and as shown in FIG. 3, the articulating joint 10 may include a first connector 35 for securing the first object 12 to stem portion 46 of the first articulating member 18 of the articulating joint 10. The first connector 35 may include a body 39 defining a first bore 43 matingly fitted with the stem portion 46 of the first articulating member 18 as well as a second bore 47 for connection with the first object 12.

Referring again to FIG. 2, the articulating joint 10 includes a second connector 51 for securing the stem portion 62 of the second articulating member 20 to the second object 14. Second connector 51 may as shown in FIG. 2 be similar or even identical to the first connector 35.

Referring now to FIG. 3A, another embodiment of the present invention is shown as articulating joint 10B. The articulating joint 10B includes an actuator 22B, which is different than the actuator 22 of the articulating joint 10. For example, as is shown in FIG. 3A, the actuator 22B includes a wedge 32B, which replaces the cam 32B of the actuator 22 of the articulating joint 10 of FIG. 2. The wedge 32B extends from shaft 30B, which is positioned in elongated opening 28B formed in body 16B of the articulating joint 10B. As the actuator 22B is advanced in the direction of arrow 74B, the wedge 32B engages with first piston 70B with second piston 72B to engage the articulating members and thereby simultaneously lock the articulating members.

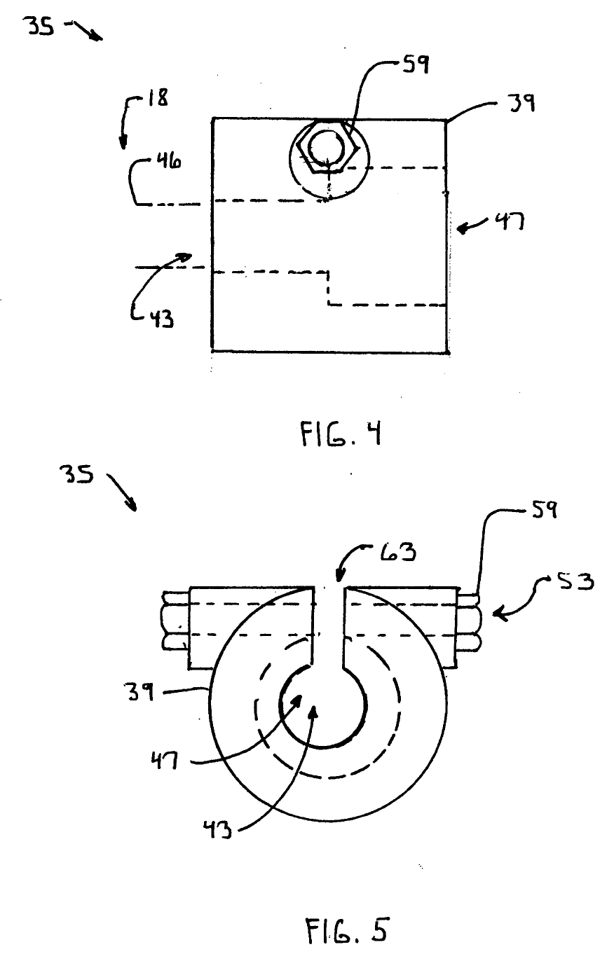

Referring now to FIGS. 4 and 5 the first connector 35 is shown in greater detail. While the first connector 35 may have any suitable size and shape, the first connector 35, as shown in FIGS. 4 and 5, includes the body 39, which has a generally hollow cylindrical shape. The body 39 of the first connector 35 may, as shown in FIGS. 4 and 5 include the first opening 43 and the second opening 47. The first opening 43 and the second opening 47 may, for simplicity, be concentric to each other and be generally cylindrical.

Referring now to FIG. 4, the body 39 may define a transverse aperture 53 for passage of a fastener in the form of bolt 59. The bolt 59 serves to compress the body 39 to secure the body 39 to the stem portion 46 of the first articulating member 18 as well as to the first object 12.

Referring now to FIG. 5, to permit the body 39 to compress against the stem portion 46 and the second object 12, the body 39 preferably includes a slit 63 extending from the opening 43 through the body 39 radially. The slit serves to provide for compression of the body 39.

The components of the articulating joint 10 of FIGS. 1-5 are preferably made of any suitable durable material that may be sterilized by commercially available sterilization techniques. For example, the components of the articulating joint 10 of the present invention may be made from a metal, a plastic, or a composite material. If made of a metal, light materials, for example aluminum, may be well suited. Composite materials, for example a carbon fiber reinforced plastic material may be well suited for components of the articulating joint 10.

Referring now to FIGS. 6 through 18, another embodiment of the present invention is shown as device 110 in the form of an actuating joint for use in trauma surgery for rigidly connecting first object 112 to second object 114.

As shown in FIG. 6, an external fixator 100 is utilized to fixably secure a first bone in the form of, for example, tibia 2 to a second bone, for example bones in the foot 4. The external device 100 includes a device 110 in the form of the actuating joint. Extending proximally from the actuating joint 110 is a first connector 135, which fixedly connects the actuating joint 110 to distractor 102.

The distractor 102 may be an optional part of the external fixator 100. The distractor 102 is utilized to distract or to draw the tibia 2 away from the foot 4. The distractor 102 serves to support a pin or, as shown in FIG. 6, a pair of spaced-apart pins or wires 104. The wires or pins 104 are inserted into tibia 2 and are used to fixedly attach the tibia 2 to the distractor 102.

Extending distally from the actuating joint 110 is a second connector 51 used to connect the device 110 to rod 106. A slit rod/pin clamp 108 is fixedly securable to the rod 106 and supports pins 104, which are secured to the bones in the foot 4.

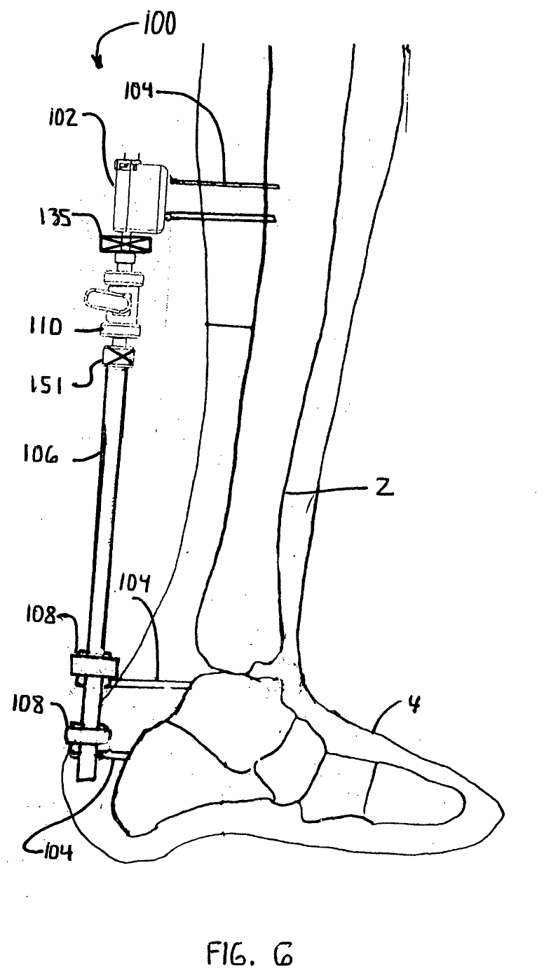

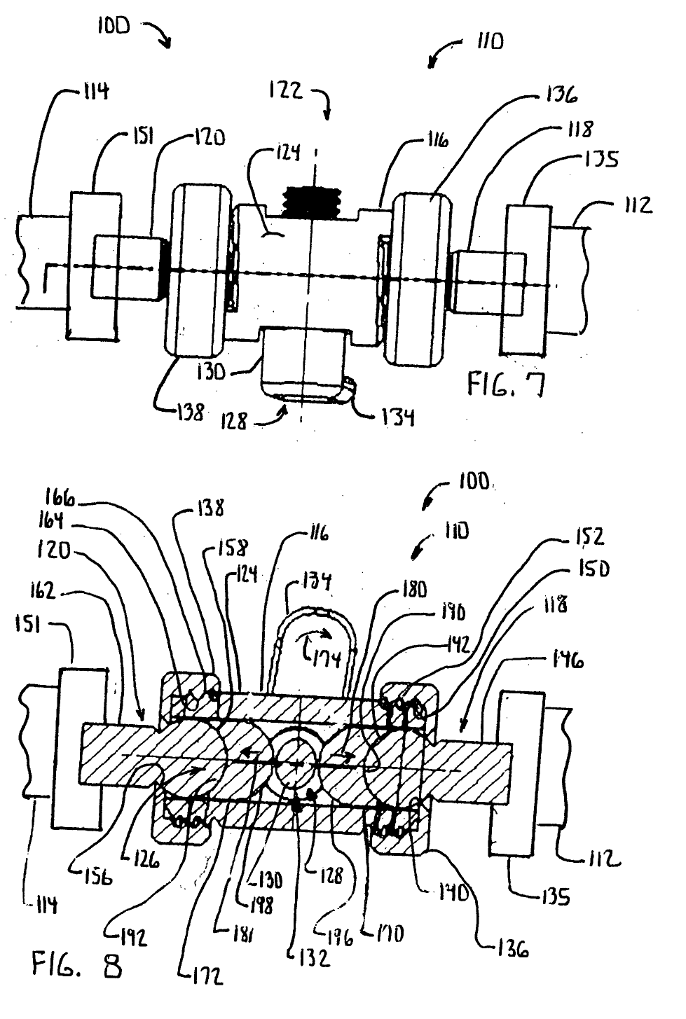

Referring now to FIG. 7, the articulating joint 110 is shown in greater detail. The articulating joint 110 may be used in the external fixator 100 for use in trauma surgery. For example, the articulating joint 110 may be a part of the fixator 100 for rigidly connecting a first object 112 to a second object 114. The articulating joint 110 of FIG. 7 is similar to the articulating joint 10 of FIG. 2. The articulating joint 110 includes a body 116 as well as a first articulating member 118 and a second articulating member 120.

The first articulating member 118 is utilized for connecting the articulating joint 110 to the first object 112. The first articulating member 118 is lockable and unlockable to the body 116 to selectively provide articulation with and rigid connection to the body 116.

The second articulating member 120 is utilized to connect the articulating joint 110 to the second object 114. The second articulating member 120 is lockable and unlockable to the body 116 to selectively provide articulation with and rigid connection to the body 116. The body 116, the first articulating member 118 and the second articulating member 120 are adapted to simultaneously lock and unlock to each other.

The articulating joint 110 may further include an actuator 122, which is operably connected to the first articulating member 118 and a second articulating member 120. The actuator 122 is utilized for simultaneously locking and unlocking of the first articulating member 118 and the second articulating member 120.

Referring now to FIG. 8, the articulating joint 110 is shown in greater detail. The body 116 of the articulating joint 110 may be of any suitable shape. The joint 110 may, as shown in FIG. 8, have a generally cylindrical body defining a cylindrical outer periphery 124 and a cylindrical bore 126 which may as shown in FIG. 8 be generally concentric with the outer periphery 124. The body 116 may further include a body transverse opening 128 formed in the body 116 for receiving the actuator 122.

The bore 126 of the body 116 is utilized to contain and guide the first articulating member 118 and the second articulating member 120. The bore 126 further contains a first piston 170 positioned between the actuator 122 and the first articulating member 118 as well as a second piston 172 positioned between the actuator 122 and the second articulating member 120.

The articulating joint 110 further includes a first cap 136 for securing the first piston 170 between cam 132 and the first articulating member 118. The first cap 136 also serves to constrain the first articulating member 118. Similarly, the articulating joint 110 further includes a second cap 138 secured to the body 116. The second cap 138 is utilized to secure the second articulating member 120 to the body 116 as well as to secure the second piston 172 between the second articulating member 120 and the cam 132.

The first cap 136 and the second cap 138 may be secured to the body 116 in any suitable fashion. For example and as shown in FIG. 8, the first cap 136 includes internal threads 150, which mate with external threads 152 formed on the body 116. Similarly, the second cap 138 includes internal threads 164, which threadably engage with external threads 166 formed on the body 116.

The articulating joint 110 is utilized to selectively lock and unlock the first articulating member 118 and the second articulating member 120 in any suitable fashion. For example, and as shown in FIG. 8, the articulating joint 110 includes the actuator 122, which may be used to actuate or selectively lock and unlock the first and second articulating members, 118 and 120, respectively. For example, and as shown in FIG. 8, the actuator 122 includes a handle 134 which may be rotated in the direction of arrow 174 to rotate the cam 132 extending from shaft 130 rotatably fitted through body transverse opening 128.

As the cam 132 rotates in the direction of arrow 174, the cam 132 contacts the first piston internal face 196 of the first piston 170 advancing it in the direction of arrow 180. Similarly, as the cam 132 is rotated, the cam contacts the second piston internal face 198 of the second piston 172 advancing it in the direction of arrow 181.

The first piston 170 as it advances in the direction of arrow 180 includes a concave outer face 190, which cooperates with spherically shaped portion 142 of the first articulating member 118 to advance the first articulating member 118 in the direction of arrow 180. As the first articulating member 118 advances in the direction of arrow 180, it contacts concave interior periphery 140 of the body 116 thereby locking the first articulating member 118.

Similarly, as the second piston 172 advances in the direction of arrow 181 outer face 192 of the piston 172 contacts spherically shaped portion 158 of the second articulating member 120 causing the second articulating member 120 to advance in the direction of arrow 181. As the spherically shaped portion 158 of the second articulating member 120 advances in the direction of arrow 181 the spherical shaped portion 158 engages with interior periphery 156 of the body 116 thereby locking the second articulating member 120 to the body 116.

The locking of the first articulating member 118 and the second articulating member 120 is accomplished simultaneously by the rotation of the handle 134 in the direction of arrow 174 by providing sufficient clearance between the shaft 130 and the body transverse opening 128 that neither the first articulating member 118 nor the second articulating member 120 are locked until all of the first articulating member 118, first piston 170, cam 132, second piston 172, and second articulating member 120 are all in mating engagement. Only then are the components subsequently locked to each other within the bore 126 of the body 116.

Extending outwardly from the first cap 136 is stem portion 146 of the first articulating member 118. The stem portion 146 is used to transfer the articulation and rigidity to the first object 112. Similarly, second stem portion 162 of the second articulating member 120 extends outwardly from the second cap 138 and is utilized to permit articulation and support to second object 114.

The first stem portion 146 and the second stem portion 162 may be operably connected to the first object 112 and the second object 114, respectively in any suitable manner. For example and as shown in FIG. 8, the first stem portion 146 may be secured to the first object 112 by a first connector 135 which is substantially similar to first connector 35 of the articulating joint 10 of FIGS. 4 and 5. Similarly, the second stem portion 160 may be secured to second object 114 by means of second connector 151, which is substantially similar to second connector 51 of FIG. 2. The first connector 135 and the second connector 151 may, for simplicity, be identical to each other.

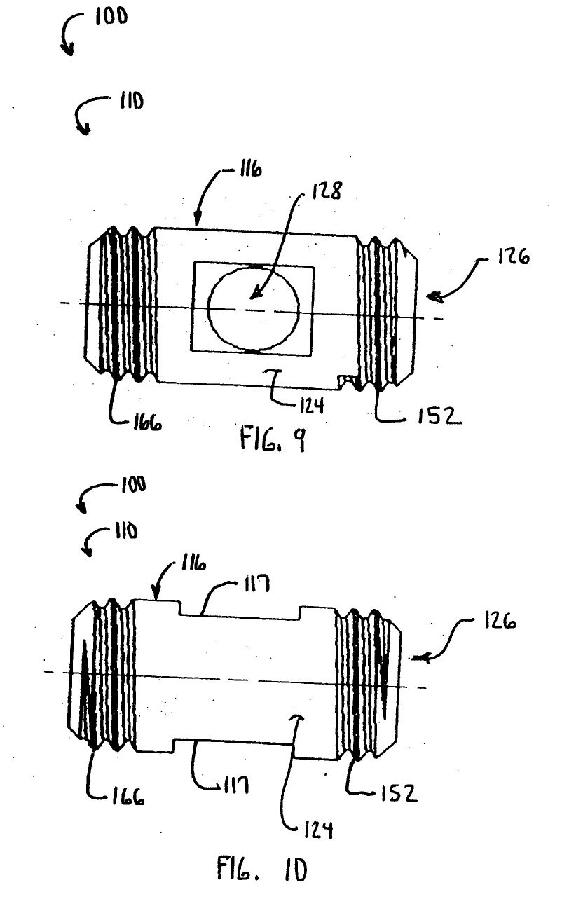

Referring now to FIGS. 9 and 10, body 116 of the articulating joint 110 is shown in greater detail. The body 116, as shown in FIGS. 9 and 10, includes the generally cylindrical outer periphery 124 as well as a generally cylindrical body bore 126, which may, as shown in FIGS. 9 and 10, be concentric with the outer periphery 124. The body 116 includes first external threads 152 and second external threads 166 formed on outer periphery 124 of the body 116.

To receive the actuator 122, the body 116 may include the transverse aperture 128 formed in the body 116. The aperture 128 may, as shown in FIG. 9, be generally oval. Alternatively, the aperture 128 may be cylindrical.

The body 116 may further include opposed parallel flats 117 positioned around the aperture 128. The flats 117 may serve to cooperate with the actuator 122.

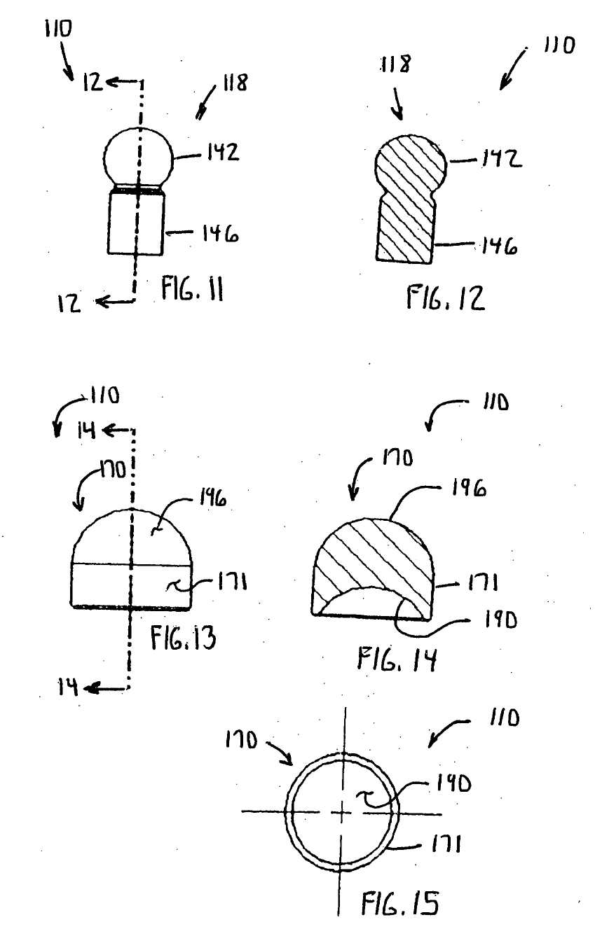

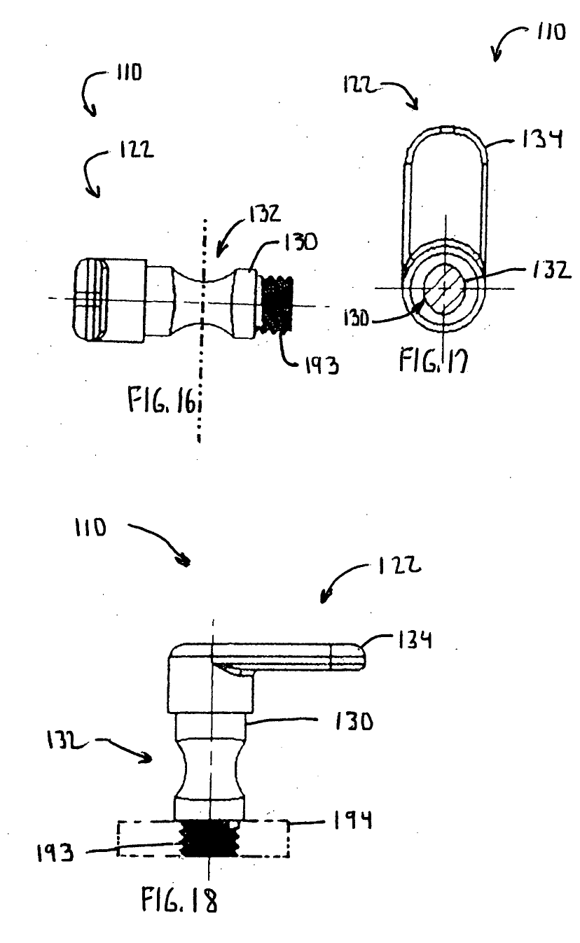

Referring now to FIGS. 11 and 12, one of the articulating members, for example first articulating member 118 of the articulating joint 110 is shown in greater detail. The first articulating member 118 may, as is shown in FIGS. 11 and 12, include an articulating portion 142, which may be as is shown in FIGS. 11 and 12 have a generally spherical periphery. Extending from the actuating or spherical portion 142 may, as shown in FIGS. 11 and 12, be a stem portion 146. As shown in FIGS. 11 and 12, the stem portion 146 is generally cylindrical. It should be appreciated that the stem portion 146 may have any suitable shape, for example, a polygonal, for example triangular, rectangular or hexagonal for mating with the connector, for example connector 135. It should be appreciated that the second articulating member 120 may have a size and shape similar or identical to the first articulating member 118.

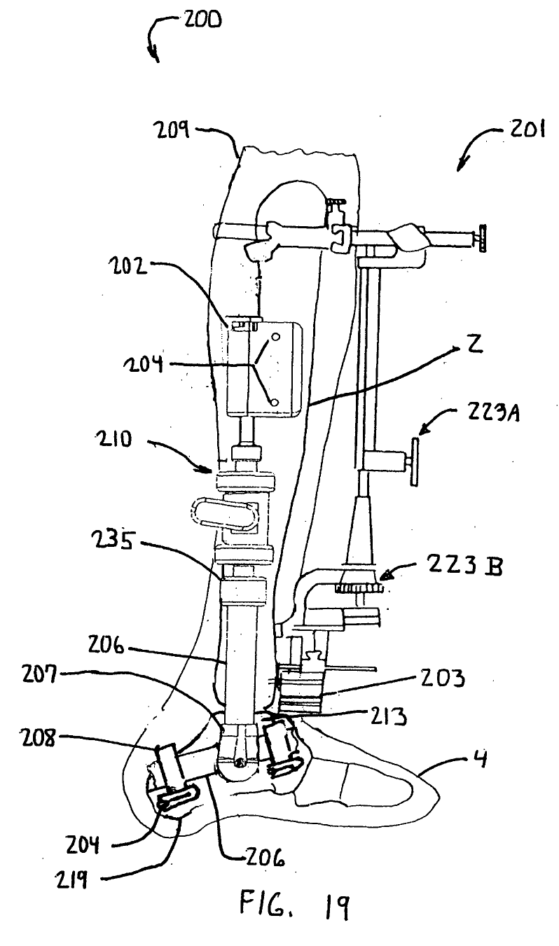

Referring now to FIGS. 13, 14 and 15, a piston for example the first piston 170 of the articulating joint 110 is shown in greater detail. First piston 170 includes an interior face 196, which serves as a follower for the cam 132 of FIG. 8. Since the piston interior face 196 serves as a follower, the interior face 196 may as shown in FIGS. 13 and 14 have a arcuate, for example a convex surface. The convex surface serves to assist in providing smooth motion with the first piston 170 with cam 132. The first piston 170 further includes an outer periphery 171, which serves as the sliding surface for cooperation with the bore 126 of the body 116 of the articulating joint 110 of FIG. 8. The outer periphery 171 preferably has a shape or contour similar to that of the bore 126.

The first piston 170 further includes an outer face 190 opposed to the interior face 196. The first outer face 190 serves to articulate with the first articulating member 118 of FIGS. 11 and 12. Since the first outer face 190 articulates with the spherical portion 142 of the first articulating member 118, first outer face 190 is preferably arcuate. For example, the first outer face 190 may be concave. For example and as shown in FIG. 14, the first outer face 190 may be in the form of a portion of a sphere.

Referring now to FIGS. 16, 17 and 18, the actuator 122 of the articulating joint 110 is shown in greater detail. The actuator 122 includes the shaft 130. The shaft 130 may have any suitable shape and may, for simplicity and as shown in FIGS. 16 to 18, have a generally cylindrical shape. It should be appreciated that the shaft 130 may be of any other shape, for example that of a polygon or any irregular shape.

The actuator 122 may as shown in FIGS. 17 and 18 further include a handle 134 extending from the shaft 130 in a direction transverse to the shaft 130. The handle 134 may have any shape and may for simplicity have a generally uniform cross-section, for example, a generally rectangular cross-section. The handle 134 may extend out sufficiently to provide the mechanical advantage necessary to lock and unlock the actuator 122.

The actuator 122 further includes cam 132. The cam 132 may have any suitable shape and may, as shown in FIGS. 16, 17, and 18, be a portion of the periphery of the shaft 130. The cam 132 may, as shown in FIGS. 16 and 18, be generally centrally located within the length of the shaft 130. The cam 132 as shown in FIG. 17 may have an oval or elliptical cross-section so that as the handle 134 is rotated the cam 132 may serve to advance the pistons 170 and 172 (see FIG. 8). The cam 132 may have a concave shape as shown in FIGS. 16 and 18 to provide a self centering feature between the cam 132 and the convex inner-faces 196 and 198 of the pistons 170 and 172, respectively.

Referring now to FIG. 18, the actuator 122 may further include a locking feature in the form of external threads 193 formed on the shaft 130 opposed to the handle 134. The external threads 193 may be used to cooperate with a fastener for example, nut 194, shown in phantom.

The articulating joint 110 and other components of the external fixator 100 of FIGS. 6-18 may be made of any suitable durable material that may be sterilized by commercially available techniques. For example the external fixator 100 and the articulating joint 110 may be made of a metal, a plastic, or a composite material. If made of a metal the articulating joint 110 is preferably made of a durable lightweight material, for example aluminum or a metal alloy, for example a cobalt chromium alloy. If the components of the articulating joint 110 are made of a plastic, the components of the articulating joint 110 are made of a durable high-strength plastic. The components of the articulating joint 110 may likewise be made of a composite material for example a carbon fiber reinforced plastic. The components to the external fixator 100 for example, the bars, may be suited for the use of carbon fiber composite materials.

Referring now to FIG. 19, yet another embodiment of the present invention is shown as external fixator 200. As shown in FIG. 19, the external fixator 200 includes an articulation joint 210 somewhat similar to the articulation joint 110 of FIGS. 6-18. In fact, the articulating joint 210 may be substantially identical to articulation joint 110 of FIGS. 6-18.

The external fixator 200 may further include a distractor 202 for use to distract the bone for example the tibia 2 from the foot 4. Wires 204 extend from distractor 202 and secure the distractor 202 to the tibia 2.

The external fixator 200 further includes a first connector 235 extending from the articulation joint 210 and opposed to the distractor 202. A carbon fiber bar 206 extends from the first connector 205. The carbon fiber bar 206 is connected to bar/end, bar clamp 207.

A second carbon fiber bar 206 extends from the bar/end bar clamp 207. The pair of bar/periphery pin clamps 208 extends in opposed direction from the bar/end, bar clamp 207 and are secured to the second carbon fiber bar 206. A series of pins 204 extend from both bar/periphery pin clamps 208 and are secured to the bones in the foot 4.

As shown in FIG. 19, a jig 201 may be used in conjunction with external fixator 200 to perform ankle fusion surgery on a patient. The jig 201 is shown in position on a leg 209 of the patient. As shown in FIG. 19, during an operation performing an ankle fusion utilizing the guide and external fixator of the present invention, an incision is made in the skin between the tibia 2 and the talus 213.

As shown in FIG. 19, the distractor 202 may be utilized or a standard ankle distractor, which is available for example, commercially from OrthoFix International NZ, Huntersville, N.C., is used to distract the ankle joint.

The ankle distractor is, for example, secured to the patient by the pins 204 which are placed in the tibia 2 and distraction pins 204 which are placed in the patient's talus 213 and calcaneous 219. The ankle distractor 202 is actuated to separate the tibia 2 from the talus 213 a distance of, for example, one centimeter.

As shown in FIG. 19, the jig 201 may include a rough adjustment mechanism 223A, which permits a cutting guide 203 to be raised and lowered vertically to roughly position the guide 203 in a location between the tibia 2 and the talus 213.

As shown in FIG. 19, the jig 201 may also include a fine-tuning adjustment mechanism 223B, which provides for precise adjustment of the guide 203 so that it may be precisely positioned between the tibia 2 and the talus 213.

Preferably, as shown in FIG. 19, the guide 203 may further include a posterior face, which preferably is positioned adjacent the interior face of the tibia 2 and adjacent the interior face of the talus 213.

When the guide 203 is properly positioned relative to the tibia 2 and the talus 213, the pins 204 are positioned in holes of the guide 203 to securely hold the guide 203 in position for the resection.

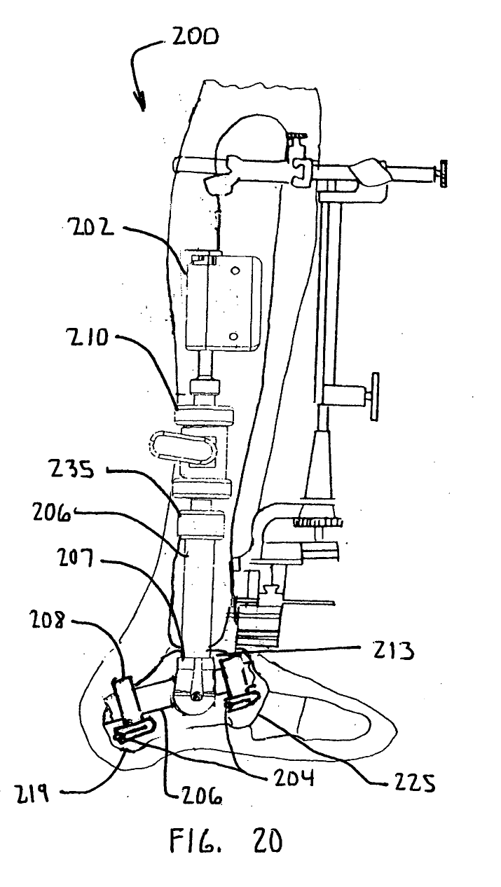

Referring now to FIG. 20, the distractor 202 and other portions of the external fixator 200 are shown in greater detail. As shown in FIG. 20, the articulating joint 210 is shown extending distally from the distractor 202. The connector 235 extends distally from the articulating joint 210. The vertical bar 206 extends vertically downward from the connector 235 and attaches to the bar/end, bar clamp 207. The horizontal bar 206 is secured to the bar/end, bar clamp 207 and is used to support the bar/periphery pin clamp 208. The bar/periphery pin clamp(s) 208 are used to secure pins 204 to the foot 4.

The pins 204 may be secured to the foot 4 in any suitable location. For example and as shown in FIG. 20, the pins 204 are secured to calcaneous 219 as well as navicular 225. It should be appreciated that other locations of the foot 4 may be used. For example and referring again to FIG. 19, the pins 204 may be secured to the calcaneous 219 as well as to the talus 213.

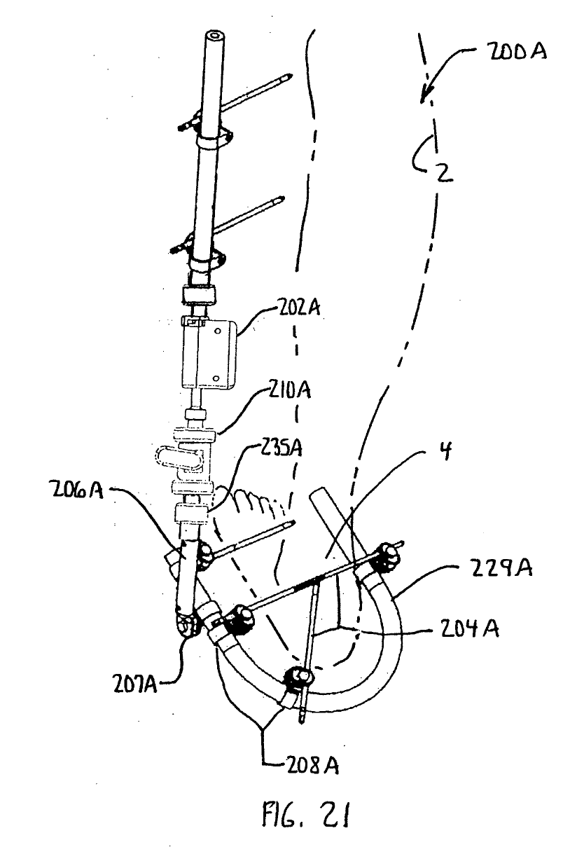

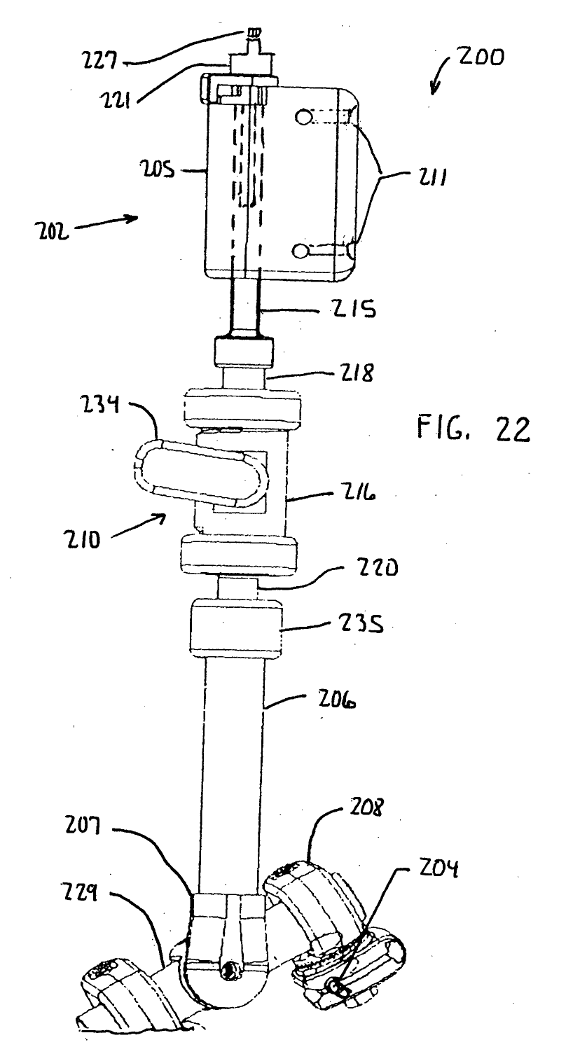

Referring now to FIG. 21 yet another embodiment of the present invention is shown as external fixator 200A. The external fixator 200A includes a distractor 202A similar to distractor 202 of FIG. 19 as well as an articulating joint 210A connected to the distractor 202A extending from the distractor 202A. The articulating joint 210A may be similar to the articulating joint 210 of FIG. 19. The external fixator 200A may further include a connector 235A connecting the articulating joint 210A to vertical bar 206A similar to the bar 206 of FIG. 20.

The external fixator 200A may further include a bar-end bar clamp 207A to connect the vertical bar 206A to arcuate bar 229A. A series of bar pin clamps 208A are located on the arcuate bar 229A and connect with various portions of the foot 4 with support pins 204A.

Referring now to FIG. 22, the external fixator 200 is shown in greater detail. The external fixator 200 includes the distractor 202. The distractor 202 may as shown in FIG. 22 include a body 205. The distractor 202 may further include a pair of fasteners in the form of screws 211 which may be threadably secured to the body 205. The screws 211 may be adapted for securing the pins 204.

The distractor 202 is operably connected to the articulating joint 210. For example, as shown in FIG. 22, the articulating joint 210 may include the first articulating member 218 for connection with the distractor 202. The first articulating member 218 may be integral with distractor pin 215. The distractor pin 215 may alternatively be a separate component from the first articulating member 218 and coupled thereto.

The distraction pin 215 is slidable fitted within the body 205 of the distractor 202. A distraction screw 221 is threadably fitted to the distraction pin 215. Distraction screw 221 includes a handle 227 which when rotated causes the distraction pin 215 and the first articulating member 218 to cause the external fixator 200 to distract.

The articulating joint 210 includes a handle 234, which rotated causes the first articulating member 218 and the second articulating member 220 to simultaneously lock with the body 205. The second articulating member 220 of the articulating joint 210 is connected to, for example, first connector 235. The first connector 235 connects the second articulating member 220 to vertical bar 206. The vertical bar 206 is connected to the bar/end bar clamp 207. The bar end/bar clamp 207 is slidably fitted to horizontal bar 229. Bar/pin clamps 208 are slidably fitted along horizontal bar 229 and receive pins 204 for cooperation with the foot 4.

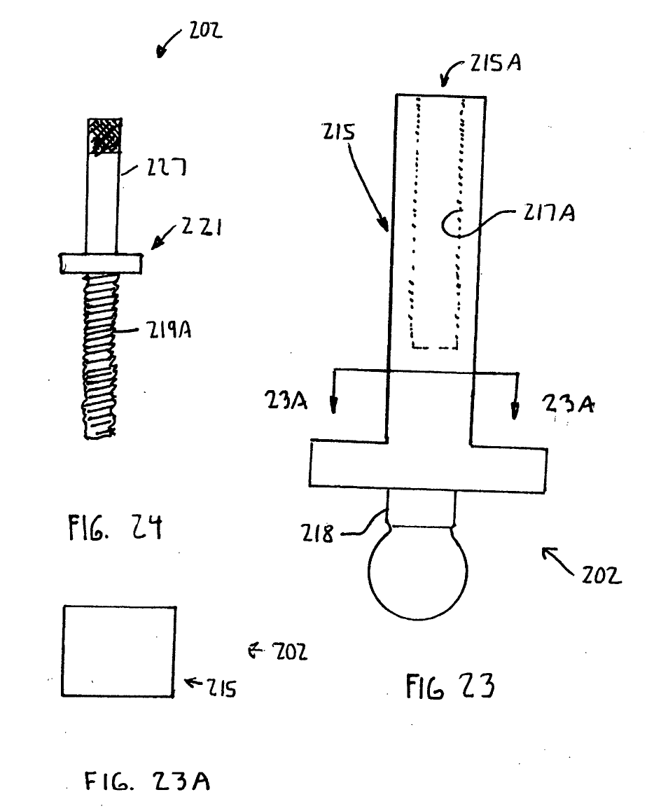

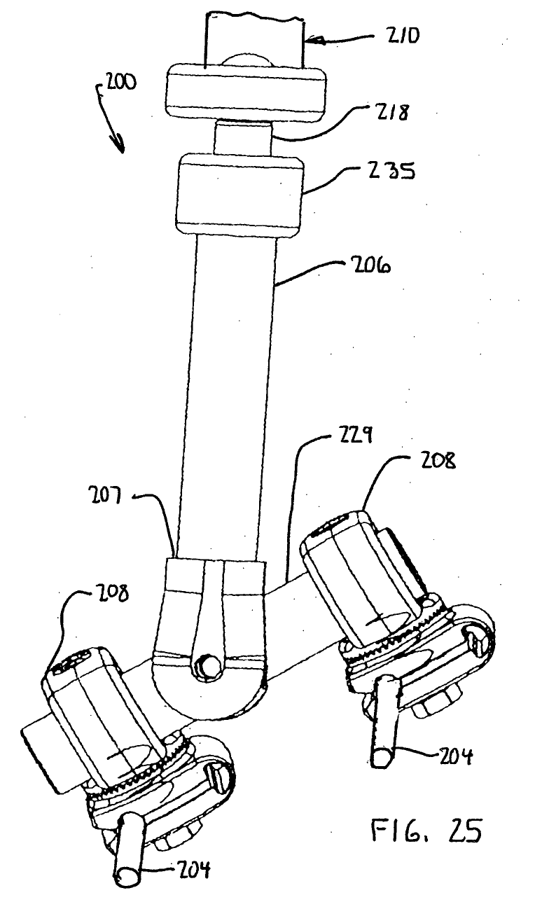

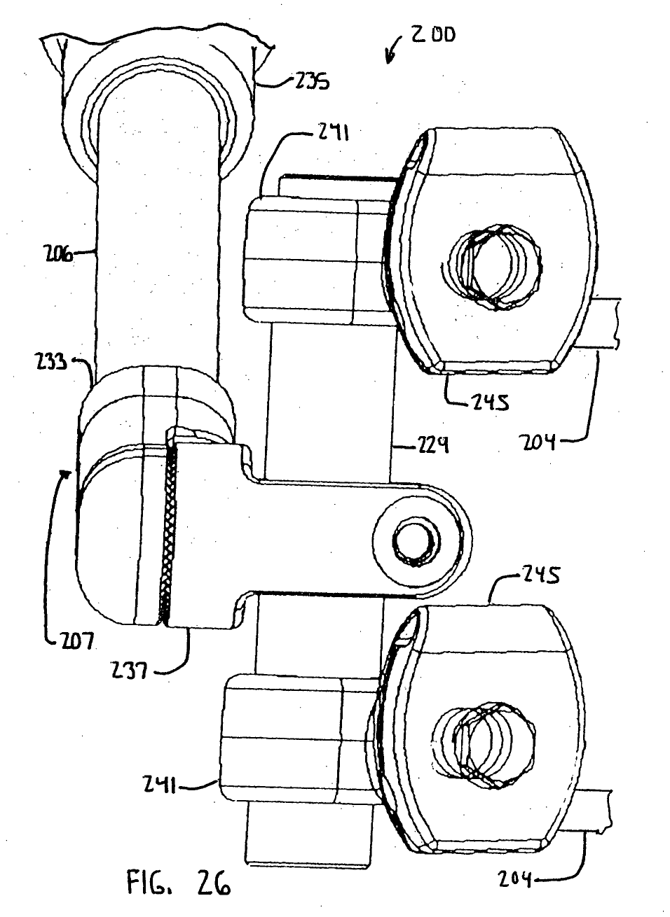

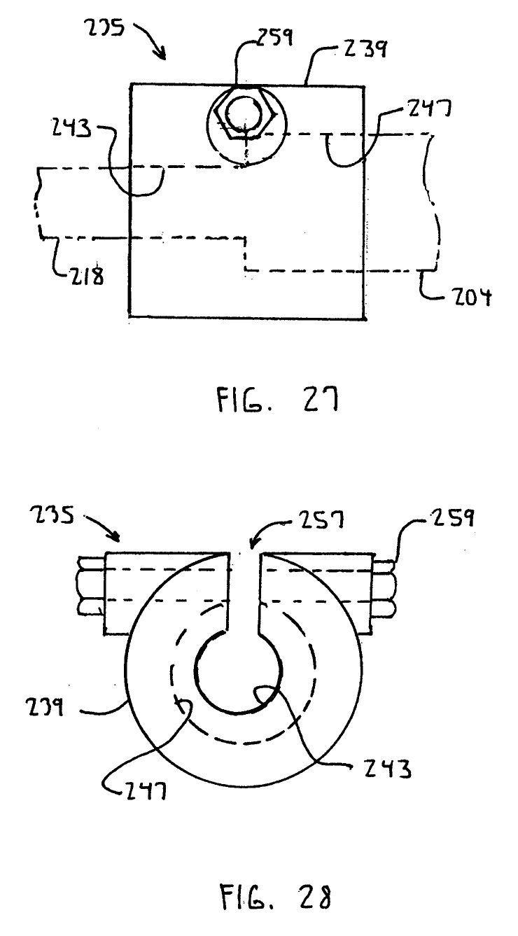

Referring now to FIGS. 23 and 23A, the distractor pin 215 is shown in greater detail. As shown in FIG. 23, the distractor pin 215 may be integral with first articulating member 218. The pin 215 may include a cavity 215A in which integral threads 217A are formed.

As shown in FIG. 23A, the pin 215 may have a rectangular or square cross-section for cooperation with the body 205 of the distractor 202 to permit translation and prevent rotation of the pin 215 when the screw 221 is rotated.

Referring now to FIG. 24, the distractor screw 221 is shown in greater detail. The screw 221 includes handle 227 for rotating the screw 221. The screw includes external threads 219A for cooperation with internal threads 217A of the pin 215.

Referring now to FIG. 25, the distal portion of the external fixator 200 is shown in greater detail. The first articulating member 218 of the articulating joint 210 is connected to first connector 235, which is connected to the vertical bar 206. The bar/end, bar clamp 207 is connected to the vertical bar 206. The bar/end, bar clamp 207 is connected to the horizontal bar 229 which receives the bar/periphery pin clamps 208, which support the pins 204.

Referring now to FIG. 26, the bars and clamps of the external fixator are shown in greater detail. The bar/end, bar clamp 207 includes a bar end half 233, which cooperates with the vertical bar 206 and a bar/periphery clamp half 237 which cooperates with the horizontal bar 229. The bar/periphery pin clamp 208 includes a bar/periphery clamp half 241 and a pin clamp half 245.

Referring now to FIG. 27, the first connector 235 is shown in greater detail. The first connector 235 includes a body 239, which defines the actuating member bore 243 and the bar bore 247. For simplicity, the bar bore 247 may be concentric with the articulating member bore 243. The articulating member bore 243 cooperates with the first articulating member 218 and the bar bore 247 cooperates with the bar 204. The body 239 includes a radial slit 257, which permits the bar bore 247 and the articulating member bore 243 to become smaller when bolt 259 is utilized to reduce the width of the slit 257.

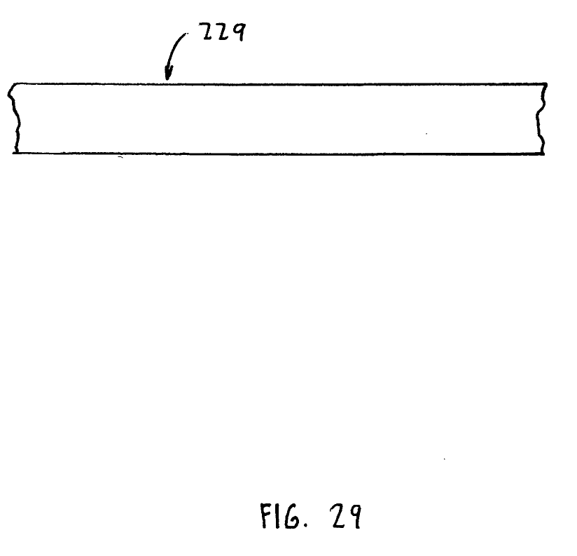

Referring now to FIG. 29, the horizontal bar 229 is shown in greater detail. The horizontal bar 229 may, for simplicity, be rectangular or in the form of a cylindrical bar. For example, as shown in FIG. 29, the bar 229 is in the form of a solid cylindrical bar.

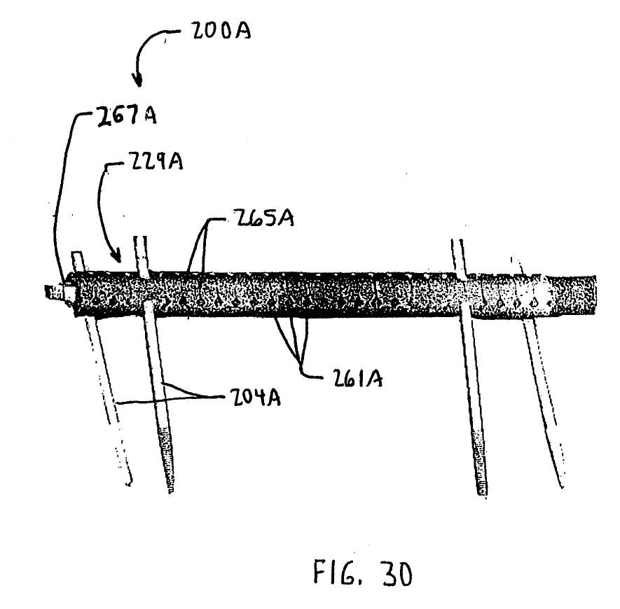

Referring now to FIG. 30, another embodiment of the present invention is shown as external fixator 200A, which is similar to the fixator 200 of FIGS. 19-30, except that the external fixator 200A includes a horizontal bar 229A, which is different than the horizontal bar 229 of FIG. 29 in that the bar 229A includes a series of cross-holes or openings 261A for receiving pins 204A. The openings 261A provide for a variety of positions for engagement of the pins 204A to the bone. Slits 257 may be positioned between the openings 261 to permit a fastener 267A to secure the pins 204A in the openings 261A.

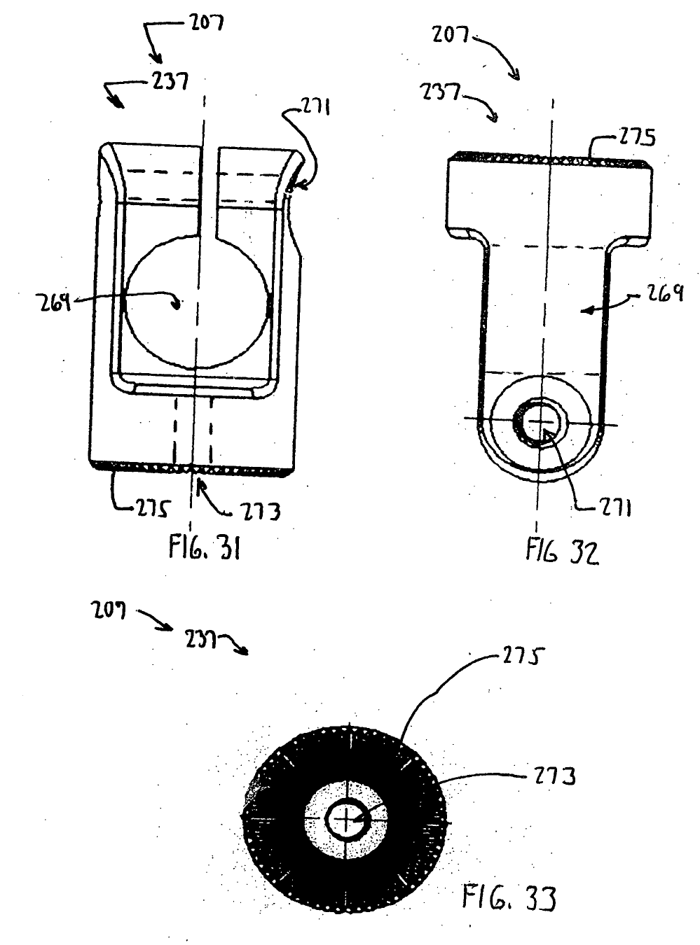

Referring now to FIGS. 31, 32 and 33, the bar/periphery clamp half 237 of the bar end/bar clamp 207 is shown in greater detail. The bar/periphery clamp half 237 includes a bar opening 269 for receiving the bar 229 (see FIG. 26) and a bar fastener opening 271 perpendicular to the bar opening 269. The bar/periphery clamp half 237 further includes a clamp fastener opening 273 for connecting the bar/periphery clamp half 237 to the bar end/bar clamp half 233. The bar/periphery clamp half 237 further includes teeth 275 for cooperation with teeth on the bar end/bar clamp half 233.

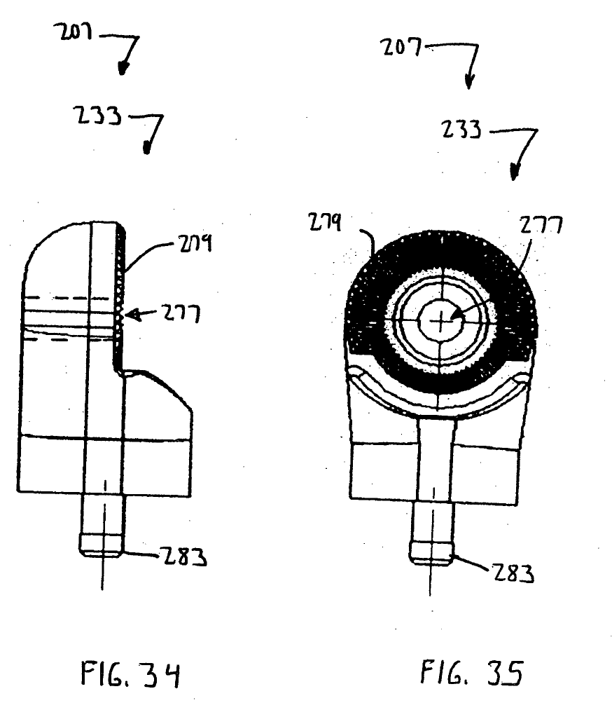

Referring now to FIGS. 34 and 35, the bar/end bar clamp half 233 of the bar end/bar clamp 207 is shown in greater detail. The bar/end clamp half 233 includes a clamp fastener opening 277 for cooperation with the bar/periphery clamp half 237. The bar/end bar clamp half 233 further includes teeth 279 for cooperation with the teeth 275 of the bar/periphery clamp half 237. The bar end clamp half 233 further includes a bar-end pin 283 for securing the bar end clamp half 233 to the vertical bar 204 (see FIG. 25).

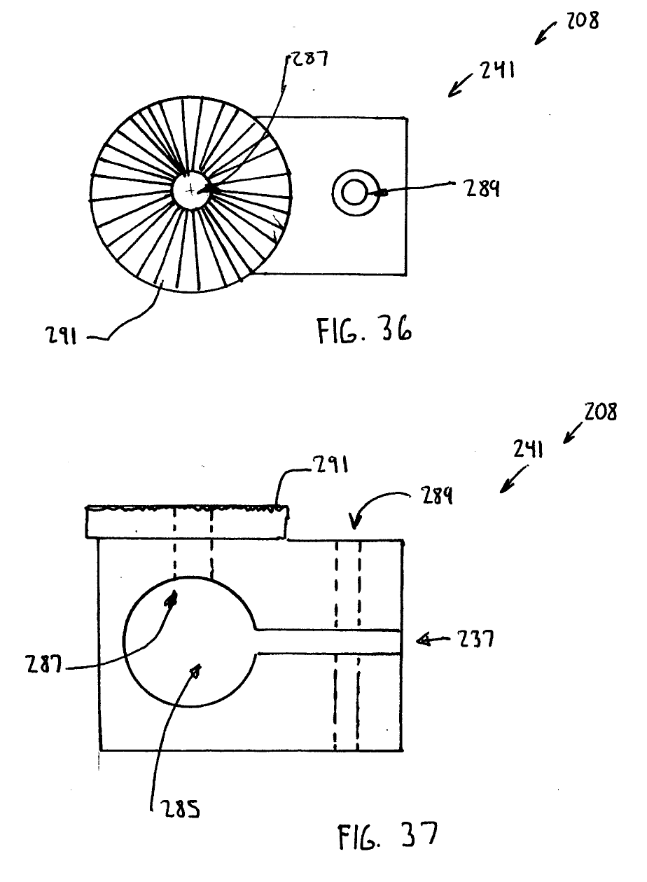

Referring now to FIGS. 36 and 37, the bar/periphery clamp half 241 of the bar/periphery pin clamp 280 is shown in greater detail. The bar/periphery clamp half 241 includes a bar opening 285 for cooperation with the bar 229 of FIG. 26. The bar/periphery clamp half 241 further includes a clamp-fastening opening 287 for connecting the bar/periphery clamp half 241 with the pin clamp half 245. The bar/periphery clamp half 241 further includes a bar fastening opening 289 for securing the bar 229 with the bar/periphery clamp half 241 with a fastener (not shown). The bar/periphery clamp half 241 may further include teeth 291 which cooperate with the pin clamp half 245.

The components of the external fixator 200 may be made of any suitable durable material, for example the components of the external fixator 200 may be made of a plastic, a metal, or a composite. If made of a metal, the components of the external fixator 200 may be made of any metal that may be sterilized by any commercially available sterilizing technique. For example, the metal components of the external fixator 200 may be made of a cobalt chromium alloy, a stainless steel alloy, or a titanium alloy. The materials of the external fixator may be made of, for example, a composite. For example, the composite material may be a carbon fiber material. The use of a carbon fiber material may reduce the weight of the external fixator. The composite material preferably is made of a sterilizable material that may be sterilized by any commercially available sterilization technique.

The materials of the external fixator 200 may, for example, be made of a plastic. If made of a plastic, the materials of the external fixator 200 should be durable and be sterilizable by commercially available techniques.

The bars, for example the vertical bar 206 and the horizontal bar 229, are well suited for the use with carbon fiber composite materials. The articulating joint 210 and the distractor 202 are adaptable for use with metal components. The clamps may be made with any suitable material, for example plastic, composites, or metals. Aluminum, because of its weight and strength, may be well suited for external fixators.

The pins 204 used in the external fixator 200 are preferably made of a material that is compatible with the human anatomy. For example, the pins 204 may be made of a metal. For example, the pins may be made of a chromium alloy, a stainless steel alloy, or a titanium alloy.

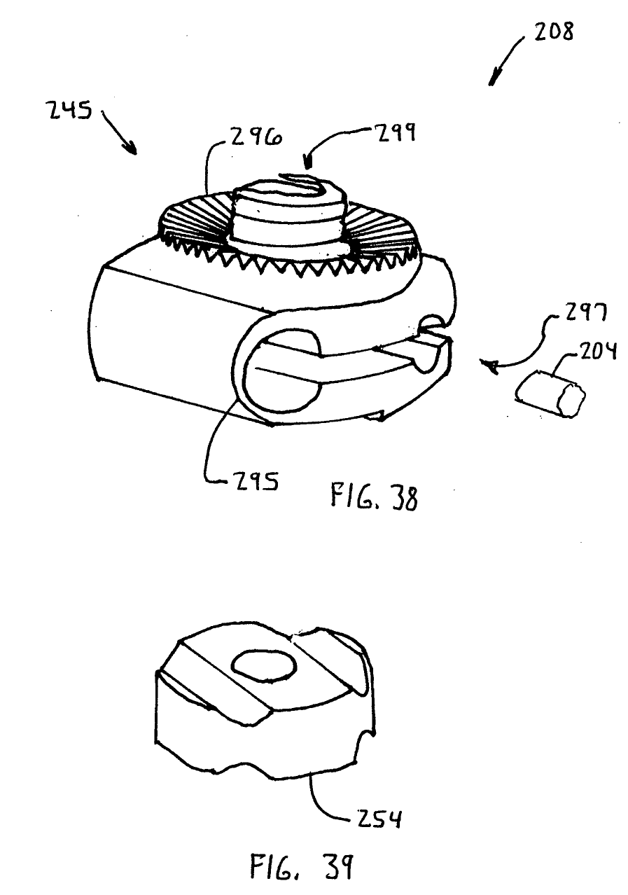

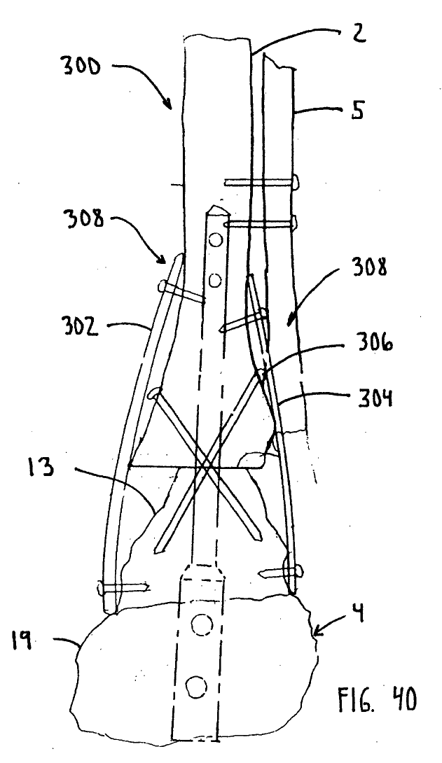

Referring now to FIGS. 38 and 39, the pin clamp half 245 of the bar/periphery pin clamp 208 is shown in greater detail. The pin clamp half 245 may be made of any suitable durable material that is sterilizable by commercially available techniques. For example, the pin clamp half 245 may be made of a plastic or a carbon fiber reinforced plastic. Alternately, the pin clamp half 245 may be made of a metal. If made of a metal the pin clamp half 245 is preferably made of a strong lightweight material, for example, aluminum. The pin clamp half 245 may be made of a plastic, for example, a nylon or another plastic. The pin clamp half 245 if made of plastic may include a living hinge 295 for assisting in clamping the wires 204. The pin clamp half 245 may include a pin opening 297 for receiving the pin 204 and a clamp fastening hole 299 for cooperation with the bar/periphery clamp half 241. The pin clamp half 245 may further include teeth 296 for engaging with the teeth 291 of the bar/periphery clamp half 241.

As shown in FIG. 39, the bar/periphery pin clamp 208 may further include a pin clamp spacer 254 positioning between the pin clamp half 245 and the bar/periphery clamp half 241 for changing the position of the pins 204 in the bone.

Referring now to FIG. 40, an implant ankle fusion set 300 is shown for use with the external fixator 200 and the jig 201 of the present invention as shown in FIG. 19. The ankle fusion implant 300 includes a lateral ankle fusion plate 302 and a medial ankle fusion plate 304. The ankle fusion plates 302 and 304 are secured to the body with screws 306 which are fitted into openings 308 in the ankle fusion plates 302 and 304.

The lateral ankle fusing plate 302 is secured to the body, for example, tibia 2, talus 13 and calcaneous 19.

The medial ankle fusion plate 304 may be connected to the tibia 2, the talus 13 and the calcaneous 19. The lateral ankle fusing plate 302 or the medial ankle fusion plate 304 or both may be connected to the fibula 5. As shown in FIG. 40, the medial ankle fusion plate 304 is connected to the fibula 5.

Referring now to FIG. 40, the ankle fusion implant set 300 may be made of any suitable durable material that are implantable in the human body, for example the ankle fusion implant sets may be made of a metal, for example cobalt chromium alloy, stainless steel alloy, or titanium alloy.

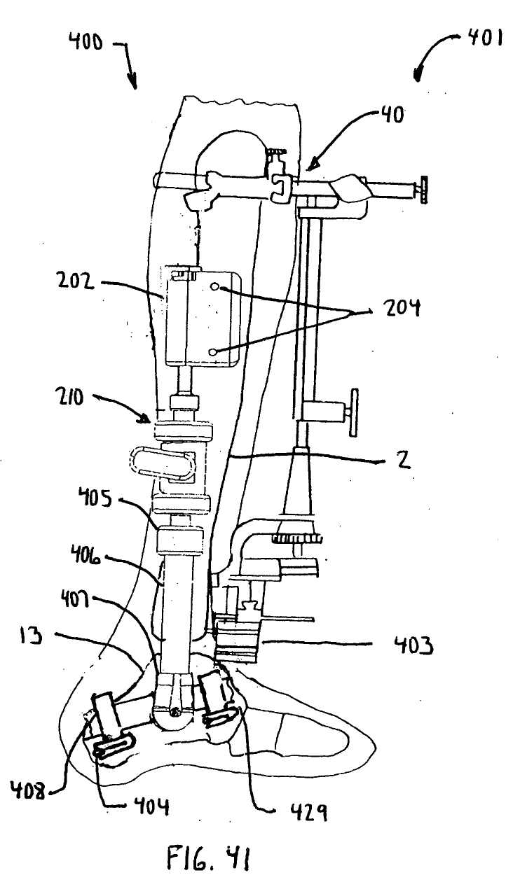

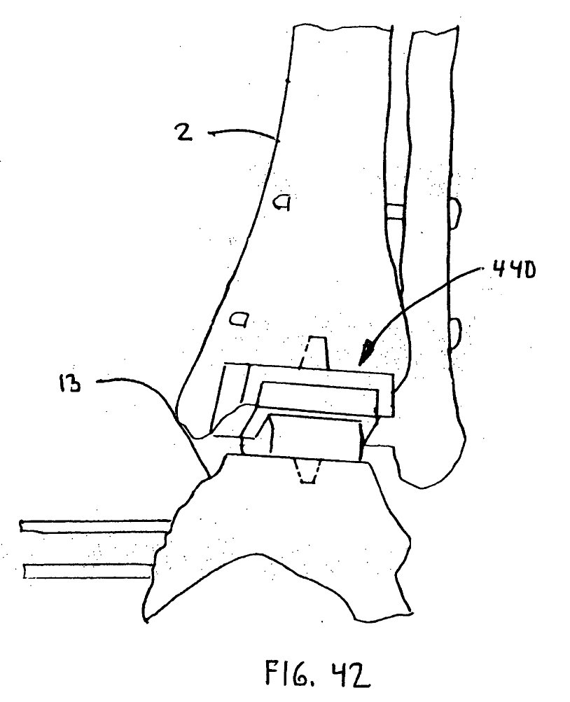

Referring now to FIG. 41 yet another embodiment of the present invention is shown as external fixator 400 for use with jig 401 to perform a total ankle arthroplasty.

The external fixator 400 includes distractor 402, which is secured by wires 404 to, for example, the tibia 2. The external fixator 400 further includes the articulating joint 210, which is connected to the distractor 202. The articulating joint 210 is connected by first connector 405 to carbon fiber bars 406 and to bar end/bar clamp 407. The bar end/bar clamp 407 is secured to horizontal bar 429. Bar/periphery pin clamps 408 are secured to the horizontal bar 429 and are used to position pins 404 to the foot 4.

The jig 401 includes a clamp 406 for securing resection guide 403 to the tibia 2. The resection guide 403 is used to resect the distal tibia 2 and the proximal talus 13. The jig 401 may be utilized for either a total ankle arthroplasty or an ankle fusion. For a patient in which total ankle arthroplasty is planned, the jig 401 may be utilized to support a resection guide 403 which is used to prepare the talus 13 and the tibia 2 for the resection cuts necessary to implant a total ankle implant.

The resection guide 403 is similar to the resection guide 203 of FIG. 19 and may include slits for performing the cuts on the tibia 2 as well as slits for performing the resection of the talus 13. The resection guide 403 may be secured, for example, to the tibia 2. The resection guide 403 may be made of any suitable durable material, for example, a metal, a cobalt chromium alloy, a stainless steel alloy, or a titanium alloy.

The jig 401 may be mounted with clamp 406 to the patient. An incision may be made in the patient and the tibia 2 and the talus 13 as well as adjacent soft tissues examined to determine whether or not a total ankle arthroplasty is advised. If the patient is a suitable candidate for total ankle arthroplasty, the resection guide 403 is mounted onto the jig 401. Conversely, if a total ankle arthroplasty is not well suited for the patient and an ankle fusion is more suited for the patient, the resection guide 203 of FIG. 19 may be utilized with the jig 201.

The external fixator 400 of FIG. 41 may be made of any suitable durable material and may, for example, be made of any materials similar to that of external fixator 200 of FIG. 19.

Referring now to FIG. 42, a total ankle implant 440 is shown in position on the patient with a portion of the total ankle implant 440 shown in position in the tibia 2 and another portion of the total ankle implant 440 positioned in the talus 13. The total ankle implant 440 is shown and explained in greater detail in U.S. Pat. No. 5,326,365 to Alvine, hereby incorporated in its entirety by reference.

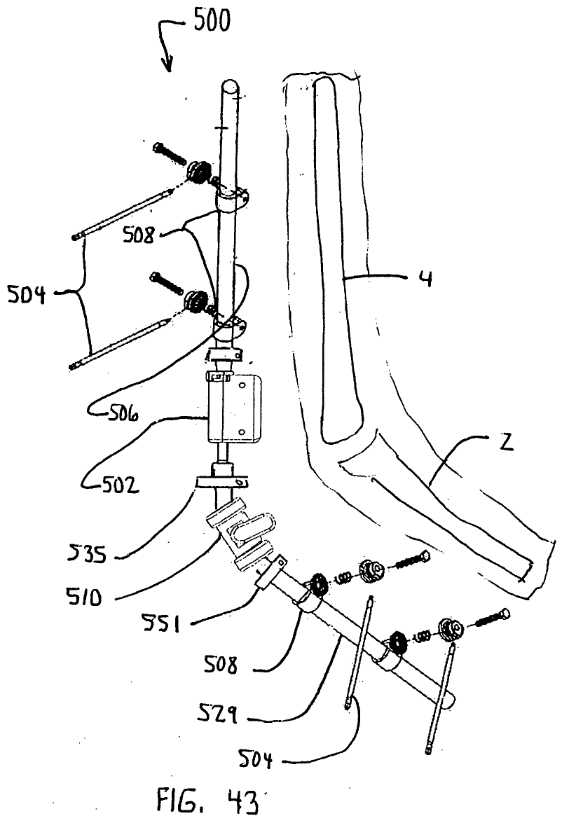

Referring now to FIG. 43, another embodiment of the present invention is shown as external fixator 500. The external fixator 500 is for use in fixedly positioning the femur 4 with respect to the tibia 2. The external fixator 500 as shown in FIG. 43 may include a pair of spaced apart bar pin clamps 508 for securing pins 504 to the femur 4. The bar pin clamps 504 are secured to femur bar 506, which is positioned spaced apart and generally parallel to the femur 4. A distractor 502 may optionally be positioned between the bar 506 and the articulating joint 510.

A first connector 535 may be utilized to connect the articulating joint 510 to the distractor 502. A second connector 551 may be positioned between the articulating joint 510 and tibia bar 529. A pair of spaced apart bar pin clamps 508 are slidably positioned on the tibia bar 529 and are used to secure pins 504 to the tibia 2. The articulating joint 510 may be used to selectively lock or position the femur 4 with respect to the tibia 2.

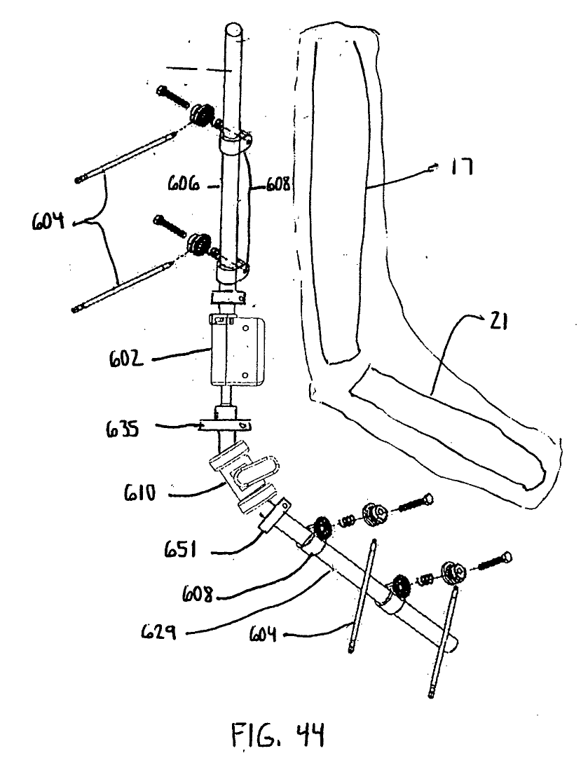

Referring now to FIG. 44, yet another embodiment of the present invention is shown as external fixator 600. The external fixator 600 may include a pair of spaced apart bar pin clamps 608, which are slidably positioned on humeral bar 606. The bar pin clamps 608 are used to secure pins 604, which are positioned in humerus 17. A distractor 602 may be positioned along the humeral bar 606 and may be connected to first connector 635, which may be positioned between the distractor 602 and articulating joint 610.

The articulating joint 610 may be connected to ulnar bar 629 by means of, for example, second connector 651. The ulnar bar 629 is used to support bar pin clamps 608, which are used to fixedly position pins 604 for securement to the ulna 21. The articulating joint 610 is used to selectively lock and unlock the external fixator 610 to lockably position the humerus 17 with respect to the ulna 21 in any of many selectable positions.

Referring now to FIGS. 43 and 44, the external fixator 500 of FIG. 43 and the external fixator 600 of FIG. 44 may be made of any suitable durable material that is sterilizable from any commonly known technique. For example, the components of the external fixator 500, as well as the external fixator 600, may be made of for example a metal, a plastic, or a composite. The pins used to secure the fixators to bone are preferably made of a material that is compatible with the human body. The pins 504 may, for example, be made of a metal, a cobalt chromium alloy, titanium alloy or stainless steel.

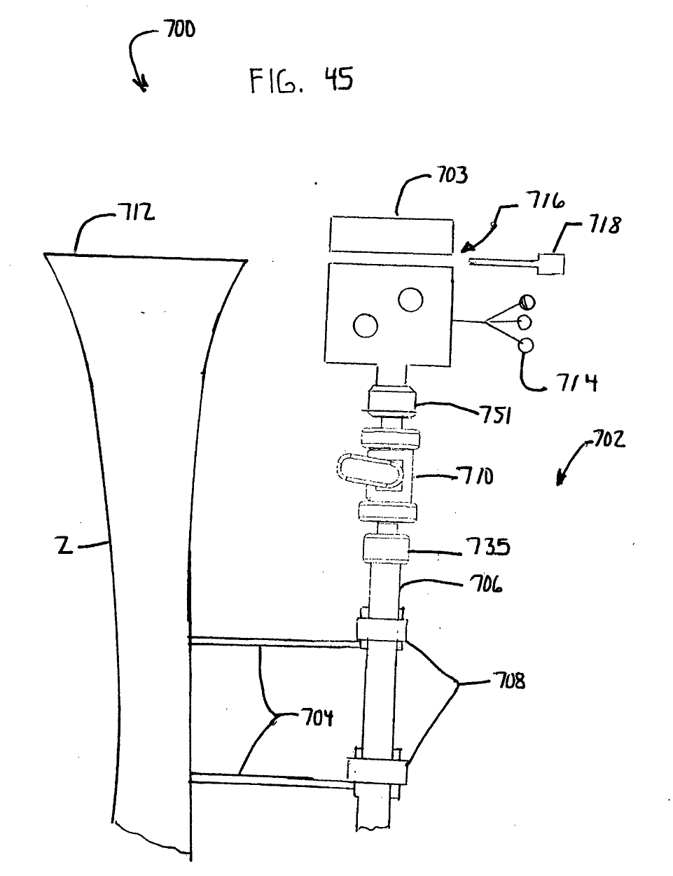

Referring now to FIGS. 45-47, the external fixator and articulating joint of the present invention may be used with a resection guide and optionally with a Computer Aided Surgery Array 714 to assist in performing arthroplasty. For example and referring now to FIG. 45, another embodiment of the present invention is shown as instrument 700. Instrument 700 is utilized to perform knee surgery, for example to prepare a resected surface 712 of the tibia 2.

The instrument 700 includes an external fixator 702, including a pair of spaced apart bar pin clamps 708, which are slidably fitted on bar 706. The bar pin clamps 708 are used to secure pins 704 for positioning in the tibia 2. The bar 706 is fixably positioned by first connector 735 to articulating joint 710. A second connector 751 is used to secure the articulating joint 710 to cutting block 703. The cutting block 703 may be selectably positioned utilizing the articulating joint 710 to properly align the cutting block with respect to the tibia 2.

A Computer Assisted Surgery (CAS) Array 714 may be utilized in conjunction with the instrument 700 for properly positioning the cutting block 703. The Computer Aided Surgery Array 714 will display the proper position of the cutting block 703. It should be appreciated that once the cutting block 703 is in the proper position the articulating joint 710 may be used to fixedly secure the cutting block 703 in that position to perform the cuts necessary to obtain the resected surface 712. The cutting block 703 may include a solitary or multiple guide slits 716 for cooperation with blade 718.

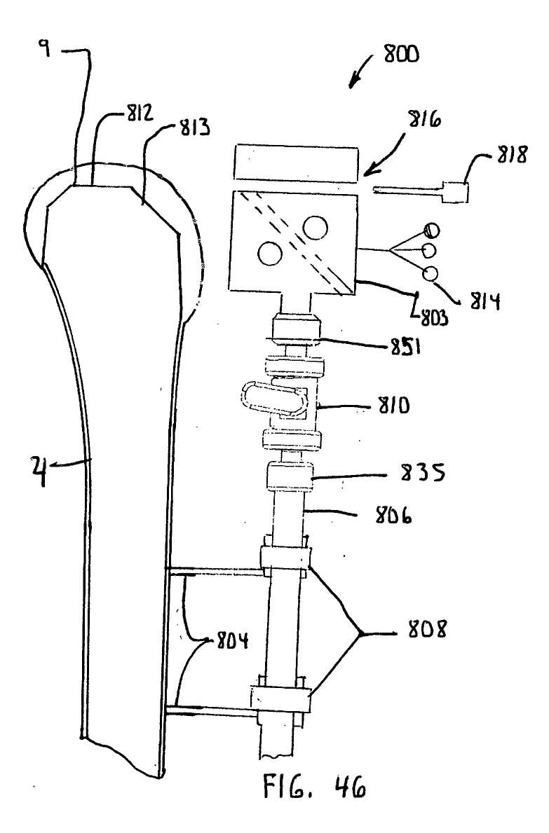

Referring now to FIG. 46, another embodiment of the present invention is shown as instrument 800 for use in preparing distal end 9 of the femur 4. The instrument 800 includes a series of spaced apart bar pin clamps 808 fixedly spacedly connected to bar 806. The bar pin clamps 808 are used to secure pins 804 to the femur 4. The bar 806 is secured by first connector 835 to articulating joint 810.

The articulating joint 810 is connected by second connector 851 to cutting block 803. The articulating joint 810 may be used to selectively lockably position the cutting block 803. The cutting block 803 may be used to perform a single or preferably multiple cuts on the distal end of the femur 4. The cuts are for preparing the knee for the femoral component of a knee prosthesis. The block 803 may include a plurality of slots 816 for preparing the resected surfaces. For example, the block 803 may be used to prepare first resection surface 812 and second resection surface 813.

The instrument 800 may further include a Computer-Aided Surgery Array 814 for use to determine the optimal position of the cutting block 803. Once the ideal position of the cutting block 803 is determined with the use of a Computer Aided Surgery Array 814, the cutting block 803 may be fixedly positioned in that position by use of the articulating joint 810. Once the ideal position of the cutting block 803 is locked into position, a blade 818 may be used in cooperation with the slits 816 to resect, for example, first surface 812.

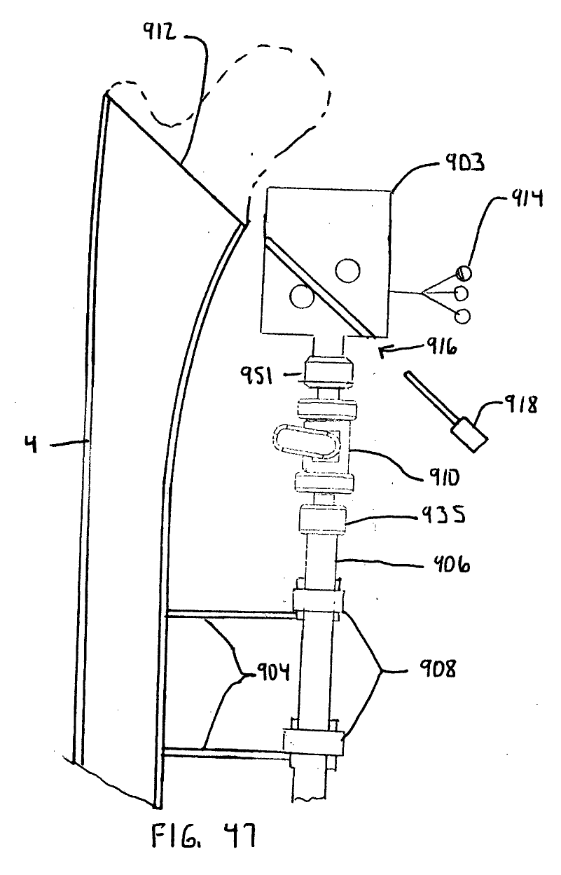

Referring now to FIG. 47, yet another embodiment of the present invention is shown as instrument 900. Instrument 900 is used to prepare resected surface 912 of the femur 4 for use in preparation for hip arthroplasty. The instrument 900 is in the form of an external fixator and includes a pair of spaced apart bar pin clamps 908, which are slidably positioned along bar 906. The bar pin clamps 908 are used to secure pins 904 to the femur 4. A first connector 935 is used to connect the bar 906 to the articulating joint 910. A second connector 951 is used to secure the articulating joint 910 to cutting block 903.

The cutting block 903 includes a slot 916 for receiving blade 918. A Computer Aided Surgery Array 914 may be secured to the cutting block 903. The Computer Aided Surgery Array 914 may be connected to computer-aided surgery equipment such that the ideal position of the cutting block 903 may be determined by use of the Computer Aided Surgery Array 914.

Once the proper position of the cutting block 903 is determined, the articulating joint 910 may be utilized to fixedly secure the cutting block 903 to the femur 4. Once the cutting block 903 is in its proper position, blade 918 may be used with the cutting block 903 to slide the blade 918 into the slit 916 to prepare resected surface 912 of the femur 4.

Referring now to FIGS. 45-47, the instrument sets 700, 800 and 900 may be made of any suitable durable material that may be readily sterilizable. For example the instruments 700, 800 or 900 may be made of a plastic, a metal, a composite material, or any combination of these materials.

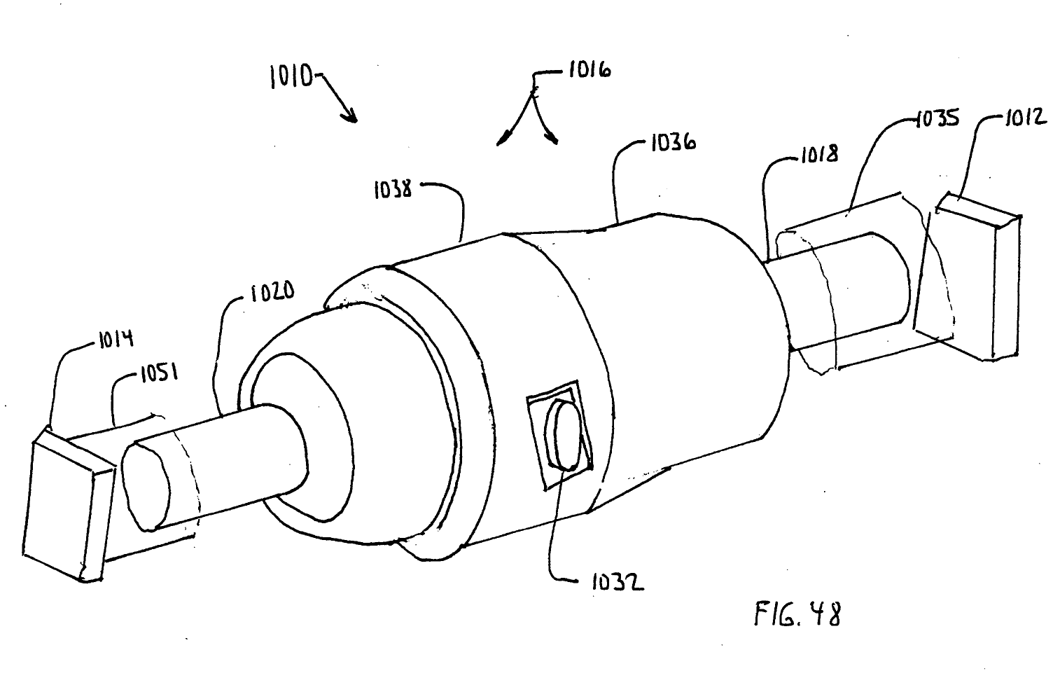

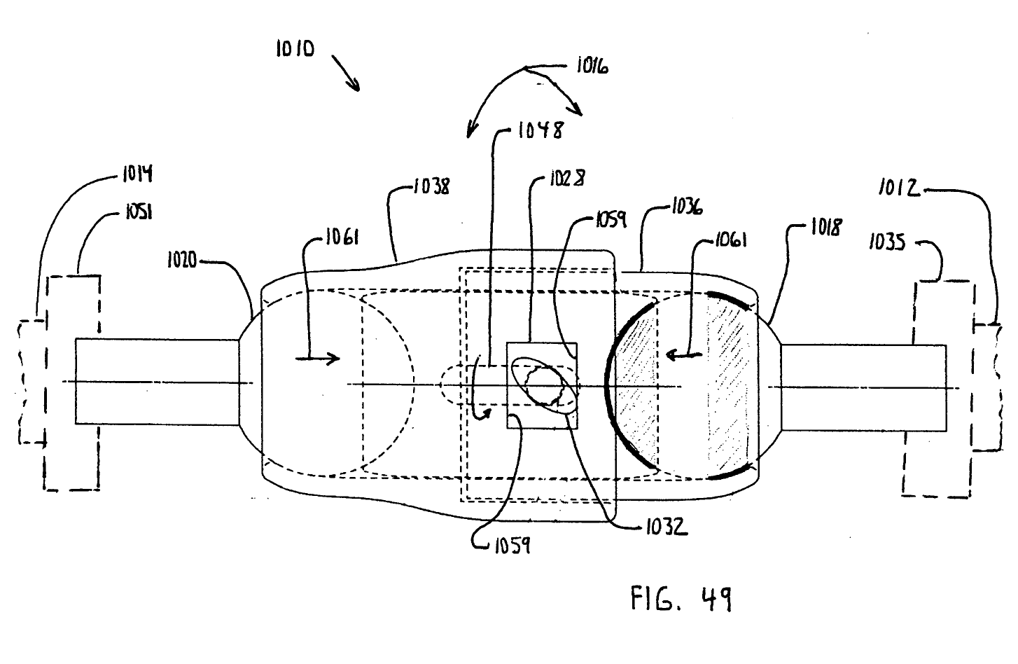

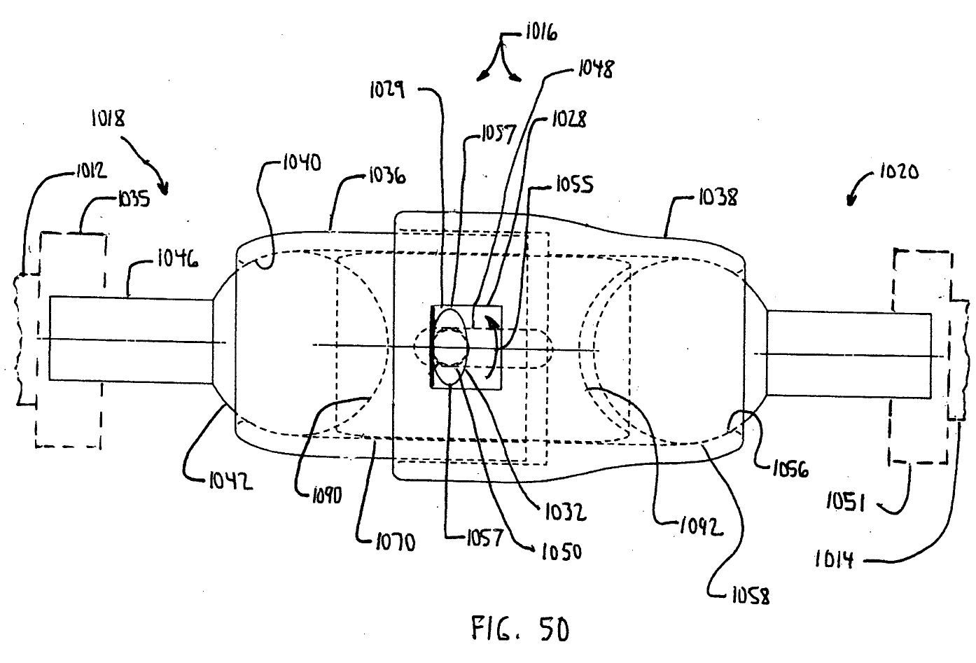

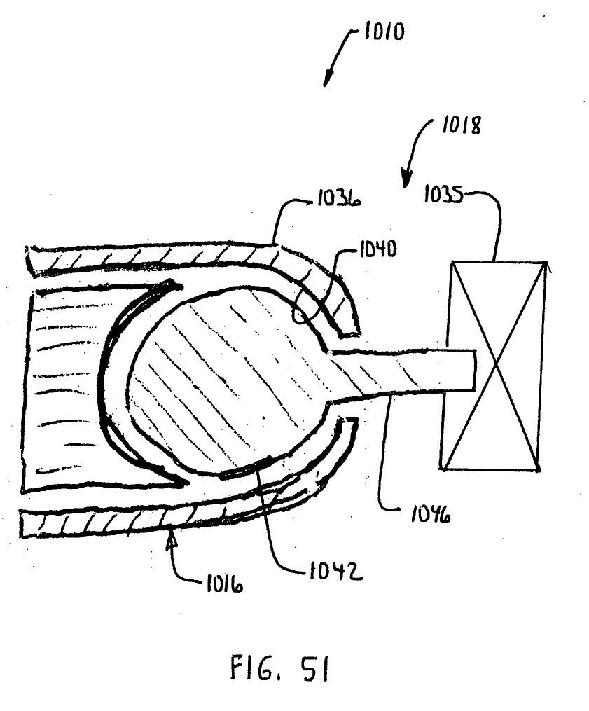

Referring now to FIGS. 48-51 yet another embodiment of the present invention is shown as articulating joint 1010. The articulating joint 1010 may be similar to the articulating joint 100 of FIGS. 6-18, but includes some modifications from the joint 100. The articulating joint 1010 includes a first cup-shaped portion 1036, which telescopes with second cup-shaped portion 1038. A solitary piston 1070 is utilized to assist in locking and unlocking the articulating joint 1010.

Referring now to FIG. 48, the articulating joint 1010 includes a body 1016 defined by the first and second cup-shaped portions 1036 and 1038, which telescopically cooperates with the first cup-shaped portion 1036. The first cup-shaped portion 1036 cooperates with first articulating member 1018. A first connector 1035 connects the first articulating member 1018 to first object 1012. The articulating joint 1010 further includes a second articulating member 1020, which cooperates with the second cup portion 1038. A second connector 1051 is utilized to connect the second articulating member 1020 to second object 1014.

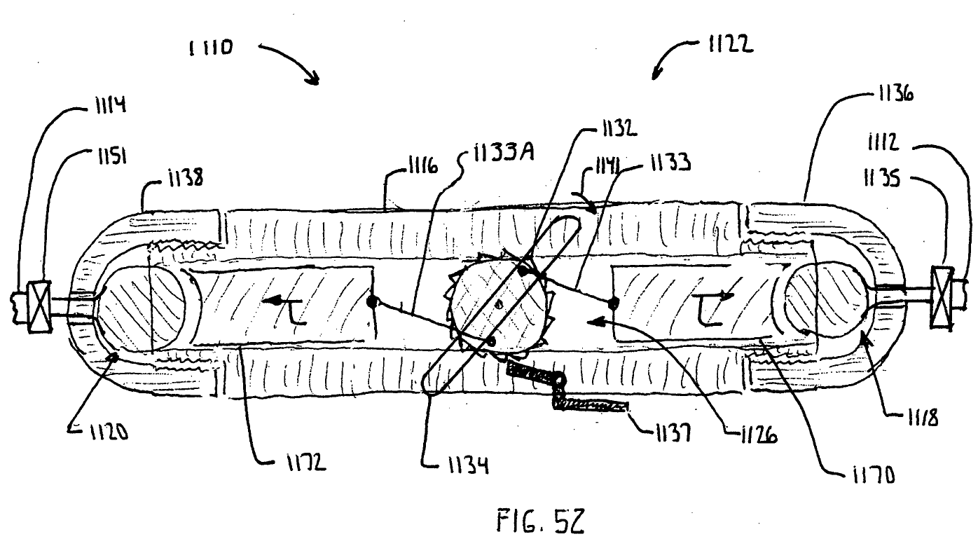

Referring now to FIG. 49, the articulating joint 1010 is shown in greater detail. As shown in FIG. 49, the body 1016 includes the first cup-shaped portion 1036 as well as the second cup-shaped portion 1038. The first cup-shaped portion 1036 is slidably fitted over piston 1070. The piston 1070, for simplicity, may have a general cylindrical shape.

The first cup-shaped portion 1036, as shown in FIG. 49, has a general cylindrical hollow shape and is slidably fitted over the piston 1070. The second cup-shaped portion 1038 is likewise slidably positioned over the piston 1070 and includes a portion of its inner-periphery that is slidably positioned over the outer periphery of the first cup-shaped portion 1036.

The first articulating member 1018 is constrained between the first cup-shaped portion 1036 and the piston 1070. Similarly, the second articulating member 1020 is constrained between the second cup-shaped portion 1038 and the piston 1070. A cam 1032 is utilized to advance and separate the first cup-shaped portion 1036 to the second cup-shaped portion 1038, thereby selectively locking and unlocking the articulating joint 1010.

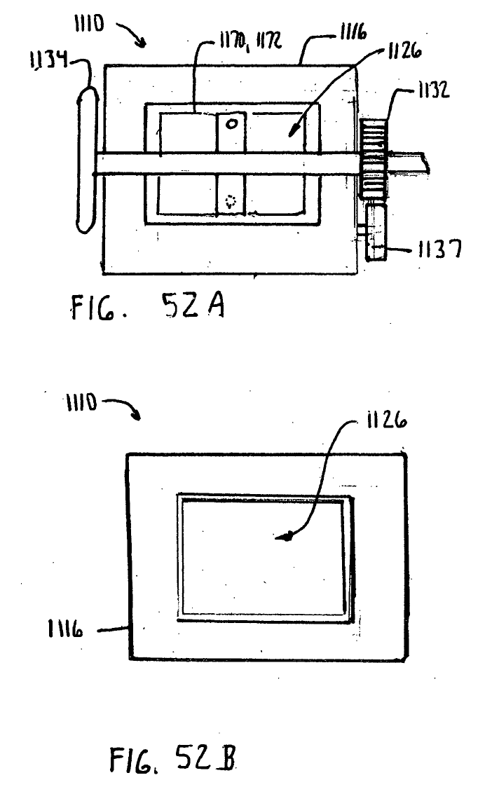

Referring now to FIGS. 50 and 51 the articulating joint 1010 is shown in an unlocked position. It should be appreciated that FIG. 49 shows the articulating joint 1010 in a locked position.

It should be appreciated that the first cup-shaped portion 1036 and the second cup-shaped portion 1038 may be selectively separated and brought together to selectively lock and unlock the articulating joint 1010 in any suitable fashion.

For example, and as shown in FIG. 51 the first cup-shaped portion 1036 may include a inner-periphery 1040, which has a concave periphery to cooperate with convex spherical portion 1042 of first articulating member 1018. The first articulating member 1018 further includes stem portion 1046 which extends from the convex spherical portion 1042 and which is secured to connector 1035. The first cup-shaped portion 1036 further includes a slot 1028 for cooperation with cam 1032.

Referring again to FIGS. 50 and 51 similarly, the second cup-shaped portion 1038 includes an inner periphery 1056, which is partially concave, for example, spherical, for mating with convex spherical portion 1058 of the second articulating member 1020. The second cup-shaped portion 1038 further includes a generally rectangular slot 1029 having a shape similar to the slot 1028 of the first cup-shaped portion 1036.

The piston 1070 has a generally solid cylindrical shape with a first concave face 1090 for cooperation with convex spherical portion 1042 of the first articulating member 1018. The piston 1070 further includes an opposed second convex face 1092 for mating cooperation with convex spherical portion 1058 of the second articulating member 1020. The piston 1070 further includes an elongated slit 1048 for cooperation with shaft 1050 connected to the cam 1032.

It should be appreciated that slot 1028 is formed in opposed sides of the first cup-shaped portion 1036. Similarly, the second slot 1029 is formed in both opposed surfaces of the second cup-shaped portion 1038. Thus, it should be appreciated that the cam 1032 is in the form of two opposed cams 1032 each fitting into a pair of the slots 1028 and 1029.

Referring now to FIGS. 49 and 50, it should be appreciated that as the cam 1032 is rotated in the direction of arrow 1055, the outer tips 1057 of the cam 1032 engages opposing faces 1059 formed from the slots 1028 and 1029 causing the first cup-shaped portion 1036 and the second cup-shaped portion 1038 to move in the direction of arrows 1061 causing the piston 1070 to cooperate with the cup-shaped portion 1036 and the second cup-shaped portion 1038 to lock the first articulating member 1018 and the second articulating member 1020 in a fixed position.

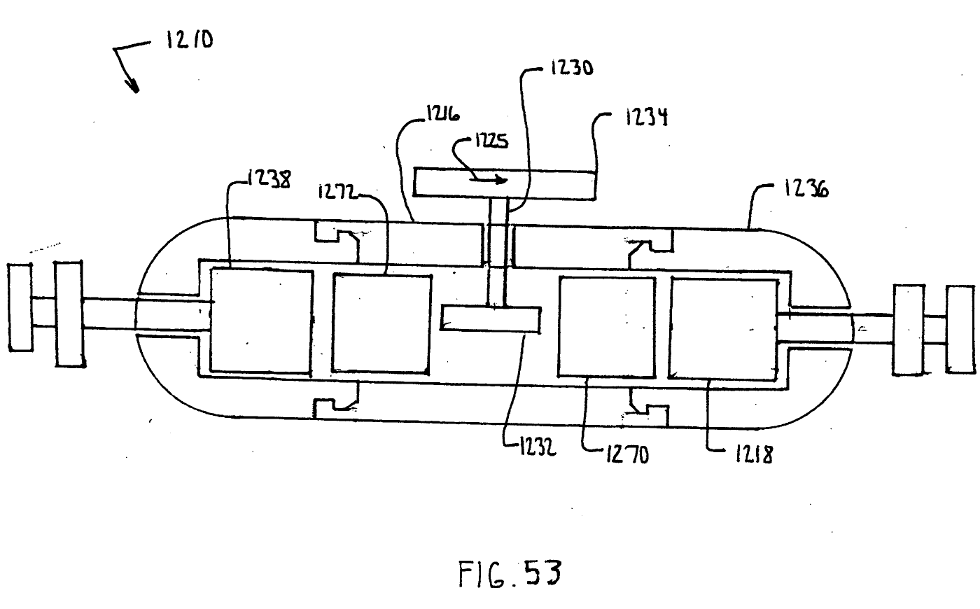

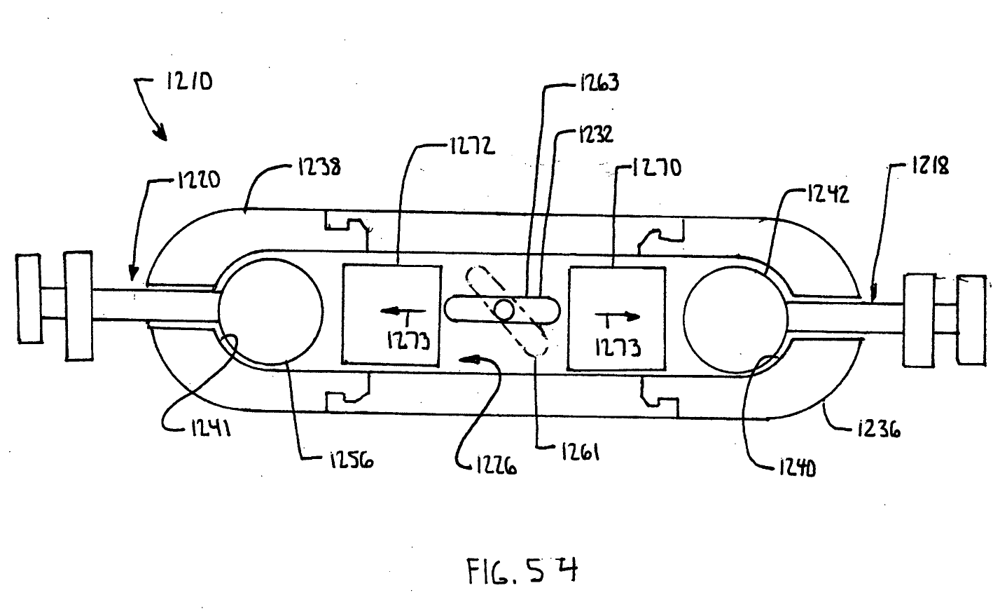

Referring now to FIGS. 52, 52A and 52B, yet another embodiment of the present invention is shown as articulating joint 1110. The articulating joint 1110 is similar to the articulating joint 100 of FIGS. 6-18, except that the cam of the articulating joint 100 of FIGS. 6-18 has been replaced by a ratchet and lever mechanism.

The articulating joint 1110 of FIGS. 50, 51 52A and 52B include a body 1116 to which a first cup-shaped portion 1136 and a second cup-shaped portion 1138 are, for example, threadedly connected. A first piston 1170 and a second piston 1172 are slidably positioned in cavity 1126 formed in the body 1116 of the articulating joint 1110. The pistons 1170 and 1172 are urged in the direction of arrows 1173 to lock the articulating joint 1110. A first articulating member 1118 is positioned between the first piston 1170 and the first cup-shaped portion 1136.

The first articulating member 1118 is fixedly connected to first object 1112 by, for example, first connector 1135. Similarly, a second articulating member 1120 is constrained between second piston 1172 and the second cup-shaped portion 1138. The second articulating member 1120 is fixedly connected to second object 1114 by second connector 1151.

The articulating joint 1110 of FIG. 52 is different than the articulating joint 10 of FIG. 1 in that the articulating joint 1110 of FIG. 52 includes an actuator 1122 in the form of a ratchet, pawl, and lever mechanism. The actuator 1122 includes a ratchet 1132, which is connected by first lever 1133 to first piston 1170 and by second lever 1133A to second piston 1172.

A pawl 1137 is pivotably connected to body 1116. Teeth 1139 formed on ratchet 1132 engage the pawl 1137. As the pawl 1137 is advanced in the direction of arrow 1179 the actuator 1122 is released, permitting the articulating joint 1110 to move freely. Extending from the ratchet 1132 is a handle 1134 that may be rotated to actuate or lock the articulating joint 1110. By rotating the handle 1134 in the direction of arrow 1141 the articulating joint 1110 may be locked.

The cavity 1126 of the body 1116 of the articulating joint 1110 may, for example, have a generally rectangular or square shape. Such shape makes possible or eases the use of the actuator 1122 including the ratchet 1132.

Referring now to FIGS. 52A and 52B, the actuator 1122 is shown in greater detail. The body 1116 of the articulating joint 1110 as shown in FIG. 52A has the cavity 1126 having a generally rectangular shape. The pistons 1170 and 1172 have a generally rectangular shape and are slidably fitted in the cavity 1126. As shown in FIG. 52A, the pistons 1170 and 1172 are slidably fitted within the rectangular cross-shaped cavity 1126. The ratchet 1132 may be positioned in the cavity 1126 and may include a portion, which extends beyond the cavity. For example, the pawl 1137 may extend outside the body 1116 so that the pawl 1137 may be actuated or released and so that the handle 1134 may be actuated with the articulating joint 1110.

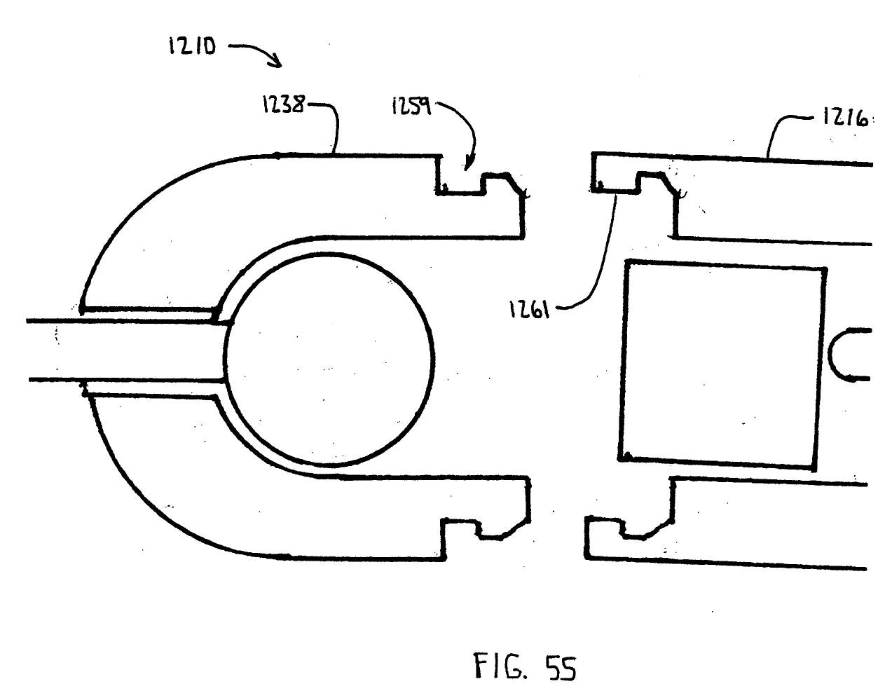

Referring now to FIGS. 53 and 54, yet another embodiment of the present invention is shown as articulating joint 1210. The articulating joint 1210 is similar to the articulating joint 10 of FIG. 1 except that the articulating joint 1210 includes first articulating member 1218 and a second articulating member 1220 which include portions that are in the form of cylinders rather than spheres.

For example and as shown in FIG. 54, the first articulating member 1218 is lockably positioned between first cap 1236 and first piston 1270. The articulating joint 1210 includes a body 1216 to which the first cap 1236 and the second cap 1238 are, for example, rotatably connected.

The body 1216 forms a longitudinal cavity 1226, which may be rectangular or have a circular cross-section. The cavity 1226 is formed to slidably receive first piston 1270 and second piston 1272. Cam 1232 is rotatably connected to the body 1216 and is utilized to advance first piston 1270 and the second piston 1272 in the direction of arrows 1273. The pistons 1270 and 1272 lock the first articulating member 1218 and the second articulating member 1220 to the first cup 1236 and the second cup 1238 respectively with respect to the body 1216. The articulating joint 1210 is thereby locked or fixed.

As shown in FIGS. 53 and 54, the first articulating member 1218 includes a cylindrical portion 1242 which mates with a cylindrical concave inner-periphery 1242 of the first cup 1236. Similarly, the second articulating member 1220 includes a cylindrical portion 1256, which matingly fits with cylindrical concave periphery 1241 formed in the second cup 1238.

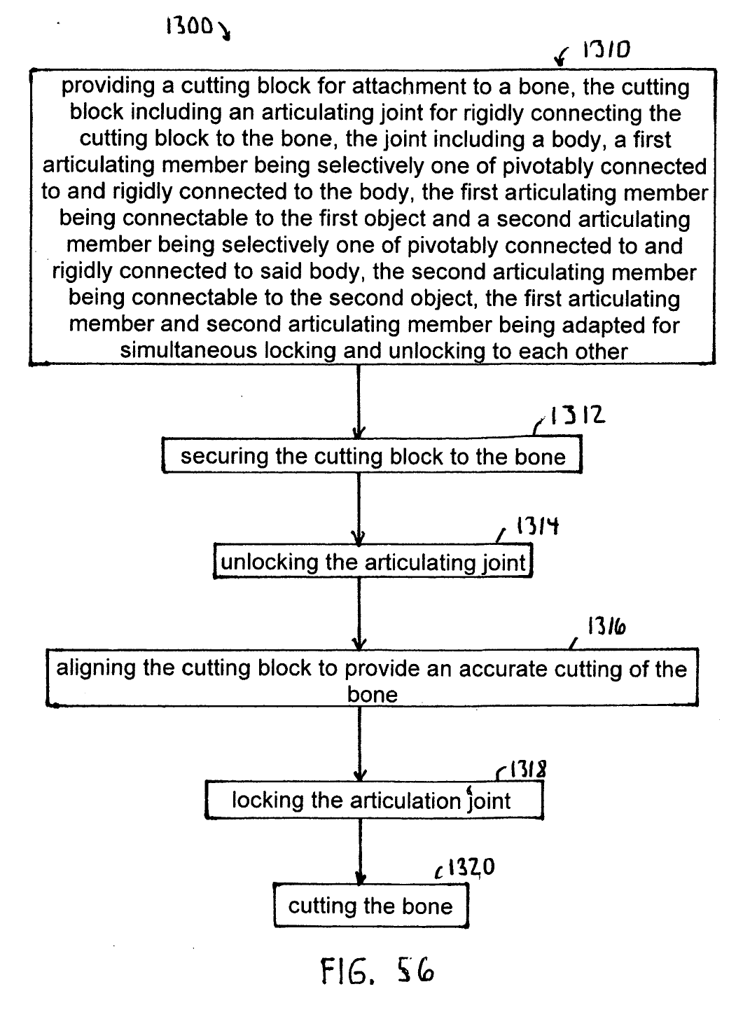

Referring now to FIG. 55, the second cup 1238 includes, for example, a groove 1259 to which a lip 1261 formed on the body 1260 matingly fit. The lip 1261 and the groove 1259 cooperate to permit the second cup 1238 to rotate with respect to the body 1216. It should be appreciated that the first cup 1236, likewise, is configured to prevent rotation of the second cup 1236 with respect to the body 1216.

Referring again to FIG. 54, as handle 1234 is rotated in the direction of arrow 1225, the cam 1232 advances toward pistons 1270 and 1272 to advance the pistons 1270 and 1272 into locking contact with the first and second articulating members 1218 and 1220.

Referring now to FIG. 54, the cam 1232 thus moves from first position 1261 (shown in phantom) to second position 1263 shown in solid. The articulating joint 1210 as shown with the cam 1232 in the second position 1263 is in a locked configuration.

Referring now to FIG. 56, yet another embodiment of the present invention is shown as surgical method or surgical procedure 1300. The surgical procedure may be used for performing arthroplasty surgery. The method 1300 includes a first step 1310 of providing a cutting block for attachment to a bone. The joint includes a body. A first articulating member may be selectively one of pivotably connected to and rigidly connected to the body.

The first articulating member is connectable to the first object and a second articulating member. The second articulating member is selectively one of pivotably and connected to and rigidly connected to the body. The second articulating member is connectable to the second object. The first articulating joint and the second articulating joint are adapted for simultaneously locking and unlocking to each other. The method 1300 further includes a second step 1312 of securing the cutting block to the bone.

The method 1300 further includes a third step 1314 of unlocking the articulating joint and a fourth step 1316 of aligning the cutting block to provide an accurate cutting of the bone. The method 1300 further includes a fifth step 1318 of locking the articulating joint and a sixth step 1320 of cutting the bone.

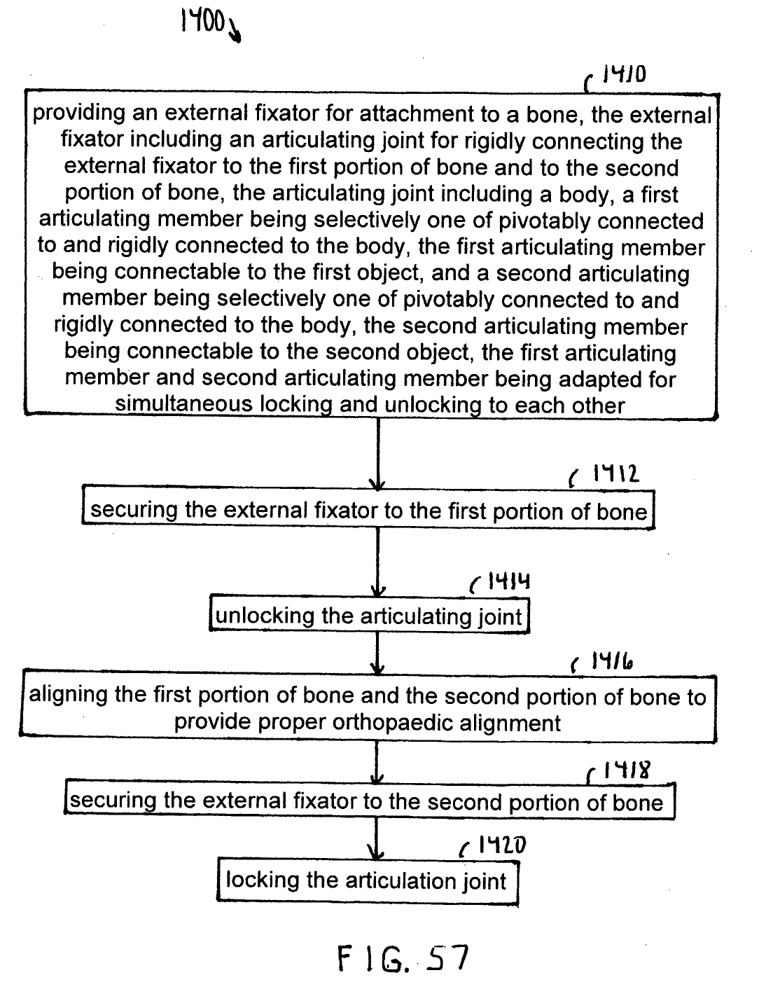

Referring now to FIG. 57, yet another embodiment of the present invention is shown as surgical procedure or surgical method 1400. The method 1400 is utilized for rigidly securing a first portion of bone to a second portion of bone during trauma surgery. The method 1400 includes a first step 1410 of providing an external fixator for attachment to the bone.

The external fixator includes an articulating joint for rigidly connecting the external fixator to the first portion of bone and to the second portion of bone. The external fixator also includes a body and a first articulating member, which is selectively one of pivotably connected to and rigidly connected to the body. The first articulating member is connectable to the first object.

The external fixator further includes a second articulating member. The second articulating member is selectively one of pivotably connected to and rigidly connected to the body. The second articulating member is connectable to the second object. The first articulating joint and the second articulating joint are adapted for simultaneously locking and unlocking to each other.

The method 1400 further includes a second step 1412 of securing the external fixator to the first portion of bone and a third step 1414 of unlocking the articulating joint. The method 1400 further includes a fourth step 1416 of aligning the first portion of bone and the second portion of bone to provide proper orthopedic alignment. The method 1400 further includes a fifth step 1418 of securing the external fixator to the second portion of bone and a sixth step 1420 of unlocking the articulating joint.

Although the present invention and its advantages have been described in detail, it should be understood that various changes, substitutions, and alterations can be made therein without departing from the spirit and scope of the present invention as defined by the appended claims.

We claim:

1. An articulating joint for rigidly connecting a first object to a second object for use in orthopaedics, comprising:
a body;
a first articulating member being selectively one of pivotably connected to and rigidly connected to said body, said first articulating member being connectable to the first object; and
a second articulating member being selectively one of pivotably connected to and rigidly connected to said body, said second articulating member being connectable to the second object, said first articulating member and second articulating member being adapted for simultaneous locking and unlocking to each other.

2. The joint as in claim 1 further comprising an actuator operably connected to said first articulating member and said second articulating member for simultaneous locking and unlocking of said first articulating member and said second articulating member.

3. The joint as in claim 1:
wherein said body defines a generally cylindrical cavity therein; and
wherein at least one of said first articulating member and said second articulating member includes a portion thereof which is generally spherically shaped for pivoting movement within the cavity of said body.

4. The joint as in claim 2, wherein said actuator includes a first portion thereof for contact with said first articulating member and a second portion thereof for contact with said second articulating member, said first portion and said second portions locking said first articulating member and said second articulating member respectively, simultaneously.

5. The joint as in claim 2, wherein said body defines an aperture therein for receiving the actuator, the aperture of said body and said actuator having sufficient clearance therebetween to permit simultaneously locking of said first articulating member and said second articulating member.

6. The joint as in claim 4, wherein the first portion and the second portion comprise a cam.

7. The joint as in claim 4, wherein the first portion and the second portion comprise a wedge.

8. The joint as in claim 1 wherein said body comprises:
a first cup-shaped portion having a closed end and an opposed open end; and
a second cup-shaped portion having a closed end and an opposed open end, a portion of the open end of the second cup slidably fitted within the open end of the first cup

9. The joint as in claim 8, further comprising a block fitted within a portion of said first cup and said second cup and operably associated with said first articulating member and said second articulating member and positioned therebetween, said first cup and said second cup being urged together to provide simultaneous locking for said first articulating member and second articulating member.

10. The joint as in claim 2, wherein said actuator comprises a member adapted to simultaneously urge said first articulating member into lockable engagement with the body and said second articulating member into lockable engagement with the body while urging said first articulating member and said second articulating member in opposed directions.

11. The joint as in claim 2:
wherein said body defines a cavity therein;
further comprising first and second pistons slidably fitted within the cavity of said body;
wherein said actuator is at least partially positioned in the cavity of said body and between said first piston and said second piston; and
wherein said actuator is adapted to simultaneously urge said first piston into lockable engagement with said first articulating member and urge said second piston into lockable engagement with said second articulating member.

12. The joint as in claim 2, wherein at least one of said pistons and at least one of said articulating members include an arcuate surface, the arcuate surface of said at least one of said articulating members conforming to the arcuate surface of said at least one of said articulating members.

13. The joint as in claim 1 wherein at least one of said articulating members comprise a periphery, a portion of which is arcuate.

14. The joint as in claim 1 wherein the portion of the periphery of said one of said articulating members is spherical.

15. The joint as in claim 1 wherein said body defines a cavity therein, the cavity being partially defined by an interior periphery of said body, a portion of said interior periphery being spherical for close conformance to the spherical portion of the periphery of said articulating member.

16. The joint as in claim 5:
wherein the aperture of said body is cylindrical; and
wherein the periphery of said actuator adjacent the aperture of said body is cylindrical.

17. The joint as in claim 5:
wherein the aperture of said body is oval; and
wherein the periphery of said actuator adjacent the aperture of said body is cylindrical.

18. The joint as in claim 1 wherein at least one of said body, said first articulating, and said second articulating member comprising one of a metal, a composite or a polymer.

19. A method for rigidly securing a first portion of bone to a second portion of bone during orthopaedic surgery, comprising the steps of:
providing an device for attachment to a bone, the device including an articulating joint for rigidly connecting the device to the first portion of bone and to the second portion of bone, the articulating joint including a body, a first articulating member being selectively one of pivotably connected to and rigidly connected to the body, the first articulating member being connectable to the first object, and a second articulating member being selectively one of pivotably connected to and rigidly connected to the body, the second articulating member being connectable to the second object, the first articulating member and second articulating member being adapted for simultaneous locking and unlocking to each other;
securing the device to the first portion of bone;
unlocking the articulating joint;
aligning the first portion of bone and the second portion of bone to provide proper orthopaedic alignment;
securing the device to the second portion of bone; and
locking the articulation joint.

20. A device for securing a first bone portion to a second portion comprising:
a first object for securement to the first bone portion;
a second object for securement to the second bone portion; and
an articulating joint for rigidly connecting said first object to said second object, said articulating joint including a first articulating member being selectively one of pivotably connected to and rigidly connected to said body, said first articulating member being connectable to the first object, and a second articulating member being selectively one of pivotably connected to and rigidly connected to said body, said second articulating member being connectable to the second object, said first articulating member and second articulating member being adapted for simultaneous locking and unlocking to each other.
